(19) United States
(12) Patent Application Publication
(10) Pub. No.: US 2026/0053863 A1
(43) Pub. Date: Feb. 26, 2026

(54) ACELLULAR ALLOGENIC EXOSOME PREPARATIONS AND METHODS OF USE

(71) Applicant: Neurocyte PTE LTD, Sydney, New South Wales (AU)

(72) Inventor: Andrew FRENCH, Sydney (AU)

(21) Appl. No.: 18/997,613

(22) PCT Filed: Jul. 27, 2023
(86) PCT No.: PCT/AU2023/050688

§ 371 Date: Jan. 22, 2025

(30) Foreign Application Priority Data
Jul. 27, 2022 (AU) 2022902095

(51) Int. Cl. A61K 35/51; A61K 9/00; A61K 45/06; A61P 25/00

(52) U.S. Cl. CPC A61K 35/51; A61K 9/0019; A61K 9/0085; A61K 45/06; A61P 25/00

(57) ABSTRACT

There is provided a method of treating or improving a symptom of a neuronal injury in a subject, the method comprising administering to the subject a therapeutically effective amount of an acellular allogenic preparation with exosomes (UCF or UCF-E) wherein the acellular allogenic preparation is derived from pooled human umbilical cord blood.

### TECHNICAL FIELD

The technology relates to acellular allogenic exosome preparations for the treatment of neuronal injuries, in particular ischemic brain injuries such as stroke or cerebrovascular accident (CVA). The technology also relates to the use of acellular allogenic exosome (extracellular vesicles (EVs)) preparations for the manufacture of a medicament for the treatment of neuronal injuries, such as stroke or CVA.

### CROSS REFERENCE TO RELATED APPLICATION

This application claims priority to Australian provisional patent application no. 2022902095, filed 27 Jul. 2022, which is incorporated by reference in its entirety.

### BACKGROUND

Globally, stroke is the second-leading cause of death and the third-leading cause of death and disability combined as expressed by disability-adjusted life-years lost (DALYs). The estimated global cost of stroke is over US$721 billion or 0.66% of the global GDP. From 1990 to 2019, the absolute number of stroke cases increased substantially (70% increase in incidence of strokes, 43% increase in deaths from stroke, 102.0% increase in prevalence of strokes, and 143.0% increase in DALYs).

A stroke is the sudden death of some brain (neural) cells due to the lack of oxygen when the blood flow to the brain is impaired by blockage or rupture of an artery to the brain. The most common types of strokes are ischemic and hemorrhagic strokes. In ischemic strokes, a lack of oxygen flow to the brain can result in apoptosis and necrosis of brain tissue leading to an infarction. Similar to cardiovascular ischemia, brain ischemia can be caused by various factors such as blood clots, thrombosis, embolism, atherosclerotic plaques, or other obstructions in the vasculature. Hypercholesterolemia, hypertension, diabetes, and obesity are risk factors for ischemic strokes. Hemorrhagic (cerebral hemorrhage) strokes are caused by a weakened vessel (artery) that ruptures and bleeds into the surrounding brain. Blood then accumulates and compresses the surrounding brain tissue and as a result the part of the body controlled by the damaged area does not function or work properly. There are two main types of hemorrhagic strokes. Intracerebral hemorrhage is when bleeding occurs inside the brain from weakened blood vessels associated with aneurysms, and arteriovenous malformation (AVMs) and Subarachnoid hemorrhage are when the bleeding occurs between the brain and the membranes that cover it.

A stroke episode, regardless of its cause, results in neural cell death, especially at the location of the obstruction or hemorrhage. In addition, biochemical reactions that occur subsequent to the stroke episode in the vasculature may lead to edema, hemorrhagic transformation, and a further compromise in neurological tissue. The neurological damage and neuron cell death that result from a stroke can be physically and mentally debilitating to the subject. Among other things, a stroke can result in problems with emotional control, awareness, sensory perception, speech, hearing, vision, cognition, movement, and mobility, and can cause paralysis.

The current treatment for ischemic stroke, tissue plasminogen activator (tPA or PLAT), while efficacious, is only effective if it is administered within three hours of the ischemic event. Therefore, treatment with tPA remains inadequate for patients who are unable to be diagnosed and treated within that time frame. tPA is also not appropriate for patients who suffer a hemorrhagic stroke, who require alternative interventional radiology or neurosurgical procedures (to stop the bleeding and reduce the pressure in the brain) as well as medications to reduce the swelling, prevent seizures and reduce pain. Considering the limitations of tPA and the high mortality rate of stroke patients, there is a clear, unmet need to reduce mortality rates, restore neural function, and extend the current window of therapeutic opportunity.

The present invention is based on the inventor's findings that acellular allogenic exosome preparations such as human umbilical cord plasma with exosomes (UCF) or exosomes only (UCF-E) derived from human umbilical cord plasma are useful in the treatment of stroke.

### SUMMARY

In a first aspect, there is provided a method of treating or improving a symptom of a neuronal injury in a subject, the method comprising administering to the subject a therapeutically effective amount of an acellular allogenic preparation with exosomes (UCF or UCF-E) consisting of acellular human umbilical cord plasma (UCF) or exosomes only (UCF-E) from human umbilical cord plasma, wherein the acellular allogenic preparation with exosomes (UCF or UCF-E) is derived from pooled human umbilical cord blood, wherein the neuronal injury is ischemic brain injury, haemorrhagic brain injury, or traumatic brain injury, and wherein the UCF or UCF-E is first administered from 3 to 90 days after the neuronal injury.

The ischemic brain injury or hemorrhagic brain injury may be a stroke.

In some embodiments an immunosuppressive agent is not administered.

In some embodiments the UCF or UCF-E is administered intrathecally.

The acellular human umbilical cord plasma preparation (UCF) and the umbilical cord plasma exosomes only preparation (UCF-E) are derived from plasma that may be characterized by a level of one or more GM-CSF, HGF, MIP-1b (CCL4) and PDGF-bb that is at least 20% higher than is found in normal adult serum.

The acellular human umbilical cord plasma preparation (UCF) and the umbilical cord plasma exosomes only preparation (UCF-E) are derived from plasma that may be characterized by a level of IP-10 (CxCL10) that is at least 60% of the level found in normal adult serum.

The acellular allogenic umbilical cord preparations with exosomes (UCF and UCF-E) may comprise exosomes with a mean, median or mode particle size of about 30-150 nm, or about 75-95 nm.

In one embodiment the acellular allogenic umbilical cord plasma with exosomes (UCF) or umbilical cord exosomes only (UCF-E) preparation is first administered from 3 to 30 days after the neuronal injury, preferably 3 to 10 days after the neuronal injury, even more preferably 7 or 8 days after the neuronal injury.

In some embodiments the acellular allogenic umbilical cord plasma with exosomes (UCF) or umbilical cord exosomes only (UCF-E) preparation is first administered from 7 to 10 days after the neuronal injury.

In some embodiments the acellular allogenic UCF or UCF-E preparation is administered periodically to the subject. For example, the acellular allogenic UCF or UCF-E preparations may be administered every week, every two weeks, every three weeks, every month or a combination thereof. Alternatively, the period of administration may be daily, every 5 days, every 10 days, every 15 days, every 20 days, every 25 days, about every 30 days, or a combination thereof.

The method may comprise administering the acellular allogenic UCF or UCF-E preparation a total of two, three, four, five, six, seven, eight, nine or ten times.

The symptom may be selected from dystonia, aphasia, infarct size, inflammation, loss of white matter, loss of grey matter, apoptotic cells in the peri-infarct region, and CD16/CD32 positive cells in the peri-infarct region.

Administration of the acellular allogenic UCF or UCF-E preparation may reduce any one or more of dystonia, aphasia, infract volume, systemic inflammation, inflammation in the infarct or peri-infarct region, the loss or gray and/or white matter in the subject.

Administration of the acellular allogenic UCF or UCF-E preparation may reduce either or both of the number of apoptotic cells in the peri-infarct region in the subject and the number of CD16/CD32 positive cells in the peri-infarct region in the subject.

The method may further comprises administering the acellular allogenic UCF or UCF-E preparation with an additional therapeutic agent such as an anti-inflammatory agent, steroid, dipyridamole, astaxanthin, dabigatran, losartan, nimodipine, policosanol, rivaroxaban, or ticlopidine.

In a second aspect there is provided a method of improving a mRS score or NIHSS score of a subject with an ischemic brain injury, the method comprising administering to the subject a therapeutically effective amount of an acellular allogenic UCF or UCF-E preparation, wherein the acellular allogenic UCF and UCF-E preparation is derived from pooled human umbilical cord blood, and wherein the UCF or UCF-E is first administered from 3 to 90 days after the ischemic brain injury.

In one embodiment an immunosuppressive agent is not administered.

The acellular allogenic human umbilical cord plasma used to isolate UCF or UCF-E may be characterized by a level of IP-10 (CxCL10) that is at least 60% of the level found in normal adult serum.

The acellular allogenic exosome preparation may comprises exosomes with a mean, median or mode particle size of about 30-150 nm.

In one embodiment the acellular allogenic plasma with exosomes (UCF) or exosome only preparation (UCF-E) is first administered from 3 to 30 days after the neuronal injury, preferably 3 to 10 days after the neuronal injury, even more preferably 7 or 8 days after the neuronal injury.

In some embodiments the acellular allogenic UCF or UCF-E is first administered 7 to 10 days after the ischemic brain injury.

In some embodiments the acellular allogenic plasma with exosomes (UCF) or exosome only (UCF-E) preparation is administered periodically to the subject. For example, the acellular allogenic exosome preparation may be administered every week, every two weeks, every three weeks, every month or a combination thereof. Alternatively, the period of administration may be daily, every 5 days, every 10 days, every 15 days, every 20 days, every 25 days, about every 30 days, or a combination thereof.

The method may comprise administering the acellular allogenic plasma with exosomes (UCF) or exosome only (UCF-E) preparation a total of two, three, four, five, six, seven, eight, nine or ten times.

In some embodiments the UCF or UCF-E is derived from blood of a single umbilical cord, or pooled blood from multiple umbilical cords. The pooled blood my be from umbilical cords with the same blood group or from umbilical cords with different blood groups.

In a third aspect there is provided a use of an acellular allogenic plasma with exosomes (UCF) or exosome only (UCF-E) preparation derived from pooled human umbilical cord blood in the manufacture of a medicament for treating or improving a symptom of a neuronal injury, wherein the neuronal injury is ischemic brain injury, haemorrhagic brain injury, or traumatic brain injury, and wherein the medicament is first administered from 3 to 90 days after the neuronal injury.

In a fourth aspect there is provided a use of an acellular allogenic plasma preparation with exosomes (UCF or UCF-E) derived from pooled human umbilical cord blood in the manufacture of a medicament for improving a mRS score or NIHSS score of a subject with an ischemic brain injury, wherein the medicament is first administered from 3 to 90 days after the ischemic brain injury.

### Definitions

Throughout this specification, unless the context clearly requires otherwise, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Throughout this specification, the term ‘consisting of’ means consisting only of.

The term “consisting essentially of” means the inclusion of the stated element(s), integer(s) or step(s), but other element(s), integer(s) or step(s) that do not materially alter or contribute to the working of the invention may also be included.

Any discussion of documents, acts, materials, devices, articles or the like which has been included in the present specification is solely for the purpose of providing a context for the present technology. It is not to be taken as an admission that any or all of these matters form part of the prior art base or were common general knowledge in the field relevant to the present technology as it existed before the priority date of each claim of this specification.

Unless the context requires otherwise or specifically stated to the contrary, integers, steps, or elements of the technology recited herein as singular integers, steps or elements clearly encompass both singular and plural forms of the recited integers, steps or elements.

In the context of the present specification, the terms ‘a’ and ‘an’ are used to refer to one or more than one (i.e., at least one) of the grammatical object of the article. By way of example, reference to ‘an element’ means one element, or more than one element.

In the context of the present specification, the term ‘about’ means that reference to a figure or value is not to be taken as an absolute figure or value, but includes margins of variation above or below the figure or value in line with what a skilled person would understand according to the art, including within typical margins of error or instrument limitation. In other words, use of the term ‘about’ is understood to refer to a range or approximation that a person or skilled in the art would consider to be equivalent to a recited value in the context of achieving the same function or result.

The terms “treating”, “treatment” and “therapy” are used herein to refer to curative therapy, prophylactic therapy, palliative therapy and preventative therapy. Thus, in the context of the present disclosure the term “treating” encompasses curing, ameliorating, or tempering the severity of an ischemic brain injury or one or more of its associated symptoms.

The terms “therapeutically effective amount” or “pharmacologically effective amount” or “effective amount” refer to an amount of an agent sufficient to produce a desired therapeutic or pharmacological effect in the subject being treated. The terms are synonymous and are intended to qualify the amount of each agent that will achieve the goal of improvement in disease severity and/or the frequency of incidence over treatment of each agent by itself while preferably avoiding or minimizing adverse side effects, including side effects typically associated with other therapies.

“Subject” includes any human and is used interchangeably with the term “patient”.

The term “neuronal injury” refers to an insult to an element of the central or peripheral nervous systems. Neuronal injuries can be derived from a physical (including mechanical, electrical or thermal), ischemic, hemorrhagic, chemical, biological or biochemical insult. Examples of neuronal injuries include ischemic and hemorrhagic stroke, spinal cord, brain, cranial nerve and peripheral nerve injuries.

In the context of this specification, the term “administering” and variations of that term including “administer” and “administration”, includes contacting, applying, delivering, or providing a compound or composition of the invention to a subject by any appropriate means.

Those skilled in the art will appreciate that the technology described herein is susceptible to variations and modifications other than those specifically described. It is to be understood that the technology includes all such variations and modifications. For the avoidance of doubt, the technology also includes all of the steps, features, and compounds referred to or indicated in this specification, individually or collectively, and any and all combinations of any two or more of said steps, features, and compounds.

In order that the present technology may be more clearly understood, preferred embodiments will be described with reference to the following description.

### FIGURES

FIG. 1: Light microscopy images of the pooled umbilical cord blood middle layer (A), top layer (B), acellular human umbilical cord plasma with exosomes (UCF) (C), and water (D).

FIG. 2: Cytokine profiles of three samples of acellular plasma compared to normal human (Adult) serum (serum-hAb (Biocore)) as a control.

FIG. 3: Samples of the starting material (acellular plasma), concentrated product (exosomes) and final product (freeze dried exosomes) characterized by particle size distribution.

FIG. 4. Modified neurological severity scores (mNSS) evaluation.

FIG. 5. Functional recovery evaluated by the cylinder test (A) and the limb placing score (B).

FIG. 6. Evaluation of infarction volume by TTC (2, 3,5-triphenyl tetrazolium chloride) staining (6A). Samples were compared with control groups. Intracranial injection (ic) groups were compared with intraperitoneally injection (ip) groups (6B).

FIG. 7. Lesion sizes at 1 d and 21 d after the experimental procedure were evaluated using magnetic resonance imaging (MRI) (7A). No statistically significant difference was found (7B).

FIG. 8. Representative photomicrographs of microtubule-associated protein 2 (MAP2) and myelin basic protein (MBP) staining at 21 days after treatment (8A). Quantification of MAP2-positive and MBP-positive area loss expressed as ratio ipsi-/contralateral area (8B). Data represents mean±SEM. Sham n=3, Control saline-ip n=3, Control saline-ic n=3, UCF-ip n=3, UCF-ic n=3, UCF-E-ip n=3, and UCF-E-ic n=3. *P <0.05 vs control saline groups, **P<0.01 vs control saline groups, **P<0.001 vs control saline groups; $P<0.05 vs between drug treatment groups.

FIG. 9. TUNEL staining quantification of apoptotic cells in the para-infarct areas 14 days after treatment.

FIG. 10. Quantification of GFAP positive cells in the para-infarct areas 14 days after treatment.

FIG. 11. Quantification of Arg-1 (11A) and CD16/32 (11B) positive cells in the para-infarct areas 14 days after treatment.

### DESCRIPTION OF EMBODIMENTS

The methods disclosed herein involve administration of acellular human umbilical cord plasma (UCF) or exosomes derived from acellular human umbilical cord plasma (UCF-E). The inventors have surprisingly found that administration of UCF or UCF-E can treat or improve one or more symptoms of a neuronal injury such as an ischemic brain jury (e.g. stroke).

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

### Exosome Preparations

Acellular plasma for use in the methods described herein can be prepared from pooled human umbilical cord blood. In particular, plasma is obtained from cord blood collected at the time of birth from normal healthy pregnancies. Plasma is not obtained from cord blood from pregnancies, that are or have been deemed high risk or which involved difficulties during childbirth. Specifically umbilical cord blood is collected aseptically and pooled before centrifugation to separate plasma, red blood cells and the buffy coat. The plasma and buffy coat were pooled and frozen at, for example, −80° C. before thawing and centrifugation at, for example, 3000 g for 20 minutes to remove cells, membranes, and cell/cellular debris. The supernatant is filtered through a 0.1 to 0.22 micron filter (with or without prefiltration for example using a 5 micron filter). The resulting hUC-P (Umbilical Cord Factor plasma) preparation, which contains exosomes, can be used immediately or lyophilized.

In some embodiments, the allogenic acellular plasma preparations (in particular UCF) are characterized by the presence of certain proteins, including at least one of IL-1 alpha, IL-1-beta, IL-1 ra, IL-2, IL-4, IL-5, IL-6, IL-7, IL-8 (CXCL8), IL-9, IL-10, IL-12 p70, IL-13, IL-15, IL-17A, eotaxin (CCL11), G-CSF, GM-CSF, FGF basic (FGF-2), HGF, IFN-gamma, IP-10 (CxCL10), MCP-1 (CCL2), MIP-1b (CCL4), PDGF-bb, RANTES (CCL5), THF-alpha, and VEGF, for example, as detected by ELISA.

In some embodiments, the allogenic acellular plasma preparations (UCF plasma) are characterized by a level of one or more of GM-CSF, HGF, MIP-1b (CCL4) and PDGF-bb that is at least 20% higher than is found in normal adult serum, for example the level may be 20%, 25%, 30%, 35%, 40%, 45%, 50%, 55%, 60%, 65%, 70%, 75%, 80%, 85%, 90%, 95%, or 100% more than is found in normal adult serum. Alternatively, the exosome preparations (UCF-E) are characterized by a level of IP-10 (CxCL10) that is at least 60% of the level found in normal adult serum. For example, the level may be 60%, 55%, 50%, 45%, 40%, 35%, 30%, 25%, 20%, 15%, 10%, or 5% of the level found in normal adult serum.

Exosomes (or extracellular vesicles (EV)) are released into the bloodstream by cells under normal and pathological conditions and carry RNA, lipids, and proteins from their host cells that can represent the molecular composition of the parental cell.

The acellular allogenic exosomes (UCF and UCF-E) originate from a variety of different cells and share common structural and functional proteins, such as Rab GTPase, SNAREs, annexins, Alix, Tsg101, and tetraspanins (CD9 and CD63) and the glycosylphosphatidylinositol-anchored molecules, flotillin, cholesterol, sphingomyelin, and hexosylceramides. Lipoprotein markers APOB (chylomicrons, VLDL, IDL, and LDL) and APOE (chylomicrons, VLDL and HDL) are used to distinguish exosomes from lipoproteins.

Exosomes can be isolated by any means known in the art including but not limited to ultracentrifugation, ultrafiltration, size exclusion chromatography, polymer-based precipitation, and affinity capture on antibody-coupled magnetic beads. For example, conventional methods of EV isolation include differential and buoyant density centrifugation, ultrafiltration, size exclusion, precipitation, and immunoaffinity separation. Size exclusion chromatography uses biofluids as a mobile phase against a porous stationary phase to differentially elute molecules with an inverse speed relation to their size. That is, larger particles will elute first, followed by smaller vesicles that will enter and flow through the pores resulting in a longer path and thus increased elution time. Differential ultracentrifugation relies on the separation of EV subpopulations via gradually higher acceleration rates. Poly-ethylene glycol (PEG)-based precipitation uses a solution to facilitate a polymer-entrapped vesicle aggregate in large numbers. Immunoaffinity capture uses antibodies targeted against exosomal surface proteins to isolate specific vesicle populations. Microfluidics technology uses chips with specific antibody-mediated binding to capture exosomes efficiently. Ultrafiltration is dependent on a filter of specific pore size that creates a vesicle-rich filtrate specific to the desired size.

The differential ultracentrifugation procedure begins with a number of cleaning spin steps designed to remove cells, cellular debris, apoptotic bodies, and microvesicles. This is done by gradually separating the pellet and supernatant at increasing speeds: 300-400×g for 10 min, then 2000×g, and finally 10,000×g, to isolate a supernatant containing a relatively high concentration of exosomes albeit still contaminated with microvesicles, lipoprotein moieties, and other protein aggregates. After this step, final exosome sedimentation occurs by spinning samples at 100,000-200,000×g for 70 mins to two hours. The pellet obtained here can be resuspended in a phosphate-buffered solution (PBS) and ultracentrifuged again, which will increase the purity, but decrease the yield of the isolated exosomes. The size of exosomes found at this level ranges from 20-250 nm and contain RNA and microRNA species, with some of the common protein markers associated with exosomes: Flotillin-1, Alix, TSG101, CD81, CD63, and CD9 among others. However, some exosomal populations may not express these proteins, e.g., CD81⁻ exosomes, and/or that these markers can be found on other types of EVs as well as exosomes.

The ultrafiltration technique relies on the use of membranes with specified pore diameters to isolate particles of a pre-determined size range. Larger particles are eliminated first by using filters with pore diameters of 0.8 and 0.45 μm, leaving a relatively exosome-rich filtrate. The exosomes obtained are defined by a maximal and minimal size range via the first and last pore filtration membrane. This protocol can be used as a complement to ultracentrifugation to separate large microvesicles and exosomes, though it can be used as a stand-alone technique.

An alternate method uses nano-ultrafiltration which relies on sequential filtrations to isolate exosomes and is known as cross-flow filtration or tangential-flow filtration. The technique commences with a dead-end filtration of the cells and their debris along with large vesicles with, for example, a 1000 nm diameter. This is followed by tangential flow-based filtration to remove contaminants (mostly proteins) with a diameter smaller than the size cut off of the membrane into a waste chamber. The filtrate, containing exosomes, is then passed repeatedly through the exclusion filter, thereby concentrating the input solution. Lastly, using a specified and consistent pore size track-etched membrane with a diameter of, for example, 50-250 nm, exosomes may be further fractionated. The recovery of exosomes is dependent on the type of filter, as different membrane types and pore sizes exist. For example, cellulose membranes with a pore size of 10 kDa often have the most efficient recovery.

Poly-ethylene glycol (PEG)-based precipitation uses an aqueous PEG solution to facilitate the formation of exosome aggregates that can then be precipitated by low-speed centrifugation, for example, at 1500×g. The isolated exosomal size range is in line with other methods such as differential ultracentrifugation.

The exosomes (UCF-E) can be used immediately or lyophilized.

In some embodiments, the allogenic acellular exosome preparations (in particular UCF-E) are characterized by the presence of certain proteins, including at least one of CD63 and TSG101 and the absence of lipoprotein markers APOB (chylomicrons, VLDL, IDL, and LDL) and APOE (chylomicrons, VLDL and HDL).

In some embodiments, the exosomes have a particle size of about 10-200 nm, for example, the particle size may be about 10 nm, 20 nm, 30 nm, 40 nm, 50 nm, 60 nm, 70 nm, 80 nm, 90 nm, 100 nm, 110 nm, 120 nm, 130 nm, 140 nm, 150 nm, 160 nm, 170 nm, 180 nm, 190 nm, or about 200 nm. Preferably, the exosomes have a particle size of about 30-150 nm, for example the particle size may be about 30 nm, 40 nm, 50 nm, 60 nm, 70 nm, 80 nm, 90 nm, 100 nm, 110 nm, 120 nm, 130 nm, 140 nm, or about 150 nm. The particle size may be a mean or median particle size. Alternatively, the particle size may be a mode (i.e. the most frequently occurring particle size in a group of particles).

In some embodiments, lyophilization does not substantially change the particle size distribution of the exosomes.

The exosome and plasma with exosomes preparations are acellular. For example, no cells are visible under light microscopy (or no viable cells or cell/cellular debris or membranes are present).

### Methods

The methods described herein involve the administration of a therapeutically effective amount of human umbilical cord plasma and exosomes (hUC-P or UCF) or exosomes only (hUC-E or UCF-E) to a subject in order to treat or improve at least one symptom of a neuronal injury.

The terms “therapeutically effective”, or “therapeutic effect”, refers to that amount of the UCF or UCF-E, which will relieve to some extent one or more of the symptoms of the neuronal injury being treated. In another embodiment, the term “therapeutically effective dose” refers to the amount of the UCF or UCF-E, when administered to subject is effective to at least partially treat a neuronal injury from which the individual is suffering, or to at least partially ameliorate or improve a symptom of such an injury. As is understood in the art, the therapeutically effective amount of the UCF or UCF-E will depend at least in part upon, the mode of administration, any carrier or vehicle employed, the specific injury, other medications taken by the subject and the specific characteristics of the subject to whom the compound is to be administered (age, weight, condition, sex, etc.).

The UCF or UCF-E can be administered parenterally or intranasally. For parenteral administration, including intravenous, intraperitoneal, intracranial, intrathecal, intramuscular, subcutaneous, or intraperitoneal administration, the UCF, UCF-E or both are formulated as an injectable solution.

The UCF, UCF-E or both are suitable for injection after the final filtration step (e.g. after filtration through a 0.1 to 0.2 micron filter). Alternatively lyophilized UCF or UCF-E can be reconstituted in any suitable vehicle for injection. Suitable vehicles include water for injection, saline, buffered saline (e.g. phosphate buffered saline or 85% phosphate buffered saline), Ringer's solution, or Ringer's solution with lactate. In some embodiments, water or 85% PBS is the preferred vehicle for reconstitution of lyophilized UCF or UCF-E.

Preferably the administration is intrathecal or intravenous.

In some embodiments, the amount of allogenic acellular plasma and/or exosome preparations (UCF or UCF-E) or both that is administered and the dosage regimen for treating a neuronal injury depends on a variety of factors, including the age, weight, sex, and medical condition of the subject, the severity of the injury, the route and frequency of administration, the particular compound employed, as well as the pharmacokinetic properties (e.g., adsorption, distribution, metabolism, excretion) of the subject, and thus may vary widely. Such treatments may be administered as often as necessary and for the period of time judged necessary by the treating physician. One skilled in the art will appreciate that the dosage regime or therapeutically effective amount of the UCF, UCF-E or both to be administered may need to be optimized for each individual. In some embodiments, the UCF or UCF-E may be administered at a dose of about 0.01 mg/kg to 100 mg/kg body weight, typically between about 0.1 mg/kg and about 50 mg/kg body weight, may be appropriate, depending on the route and frequency of administration.

The dose to be administered, will preferably be in the form of single bolus, or infusion of the UCF, UCF-E or both over a period of time, periodic dosing or a combination thereof. The concentration of any given dose will depend on the frequency of administration.

In some embodiments, the UCF or UCF-E is administered periodically, for example every week, every two weeks, every three weeks, every month or a combination thereof. Alternatively, the UCF or UCF-E can be administered daily, every 5 days, every 10 days, every 15 days, every 20 days, every 25 days, about every 30 days, or a combination thereof.

In one embodiment, the treatment of a neuronal injury comprises administering the UCF, UCF-E or both multiple times for example two, three, four, five, six, seven, eight, nine or ten times, or more.

In some embodiments, the treatment begins with a first administration of the UCF, UCF-E or both at least 1 day after the neuronal injury. For example, the treatment can begin about 1, 2, 3, 4, 5, 6, 7, 8, 9, 10, 11, 12, 13, 14, 15, 16, 17, 18, 19, 20, 21, 22, 23, 24, 25, 26, 27, 28, 29, about 30, about 60 days or at least about 90 days after the neuronal injury.

In some embodiments, the treatment begins with a first administration of the UCF, UCF-E or both approximately 3, 4, 5, 6, 7, 8, 9, or 10 days after injury, for example 7 or 8 days after injury.

The UCF and/or UCF-E may be administered with additional therapeutic agents such as anti-inflammatory agents, steroids, dipyridamole, astaxanthin, dabigatran, losartan, nimodipine, policosanol, rivaroxaban, or ticlopidine.

In some embodiments, the UCF and/or UCF-E may be administered with one or more therapeutic agents that temporarily disrupt the blood-brain barrier as blood-brain barrier disruption (BBBD) is effective and safe for delivery of therapeutics. Suitable therapeutics for BBBD include one or more of mannitol, intracarotid arterial hyperosmolar mannitol (ICAHM), RMP-7 (a synthetic analog of bradykinin), and regadenoson.

In one embodiment, the UCF, UCF-E or both are not administered with an immunosuppressant.

The UCF and UCF-E described herein are useful for the treatment of a range of neuronal injuries including ischemic brain injury, hemorrhagic brain injury, or traumatic brain injury.

In particular, the UCF and UCF-E described herein are useful for the treatment of ischemic or hemorrhagic stroke.

The treatments can result in improvement of one or more symptoms of a neuronal injury. For example, the treatment may improve one or more of the following:
Dystonia—where an improvement is a reduction in the frequency or severity of involuntary muscle contractions compared to the same patient before treatment or a control patient.
Aphasia—where an improvement is an increased ability to comprehend or formulate language compared to the same patient before treatment or a control patient.
Infract size—where an improvement is a decrease in the infarct size.
Inflammation—where an improvement is a decreased inflammation either systemically or in the infarct region.
Loss of white matter—where an improvement is a reduction in the amount of white matter loss as a result of the injury.
Loss of grey matter—where an improvement is a reduction in the amount of grey matter loss as a result of the injury.
Apoptotic cells in the peri-infarct region—where an improvement is a reduction in the number of apoptotic cells in the peri-infarct region.
CD16/CD32 positive cells in the peri-infarct region—where an improvement is a reduction in the number of CD16/CD32 positive cells in the peri-infarct region.

Dystonia is typically considered a movement disorder characterized by motor manifestations, primarily sustained or intermittent muscle contractions causing abnormal, often repetitive, movements, postures, or both. However, growing evidence indicates the importance of non-motor components to dystonia, including abnormalities in sensory and perceptual functions, as well as neuropsychiatric, cognitive and sleep domains. Dystonia can be assessed by any means known in the art. This may include use of the Unified Dystonia Rating Scale (UDRS) which includes a detailed assessment of the severity of dystonia in individual body areas. For example, each body region is rated for dystonia severity and duration. The proximal and distal limbs are given separate ratings. Ratings for each body region are totaled for an overall rating of dystonia severity. Accordingly, the methods described herein can result in an improved UDRS.

Aphasia is an inability, or impaired ability, to understand or produce speech, particularly as a result of neuronal injury (such as a stroke). Aphasia can be assessed informally (via interaction and observation) by a treating physician or can be assessed using one or a combination of known tests. These tests include the Psycholinguistic Assessments of Language Processing (PALPA), the Western Aphasia Battery, the Boston Naming Test, the Boston Diagnostic Aphasia examination, the Aachen Aphasia Test, the Minnesota Test for Differential Diagnosis of Aphasia, Acute Aphasia Screening Protocol, the Mississippi Aphasia Screening Test, the Mount Wilga High Level Language Screening Test, Aphasia Language Performance scales, the NIH Stroke Scale, the Bedside Evaluation Screening Test, the Boston Diagnostic Aphasia Examination, the Porch Index of Communicative Ability, the Burden of Stroke Scale, Pyramids and Palm Trees, Caulfield Language for Cognition, Quick Assessment for Aphasia, Cognitive Linguistic Quick Test, Reitan-Indiana Aphasia Screening Examination, Communication Activities for Daily living, ScreeLing, Communicative Effectiveness Index, Sheffield Screening Test for Language Disorders, Comprehensive Aphasia Test, Sklar Aphasia Scale, Frenchay Aphasia Screening Test, Test for Reception of Grammar, Functional Assessment of Communication Skills for Adults, The Aphasia Screening Test, Functional Communication Profile, Ullevaal Aphasia Screening Test, Wechsler Individual Achievement Test, Information Language Processing Screen (ILPS), Western Aphasia Battery, Inpatient Functional Communication interview, Whurr Aphasic Screening Test, Measure of Cognitive-Linguistic Abilities. The methods described herein can result in an improved performance on any one of or any combination of these tests.

Infarct size (volume) is typically assessed using MRI or CT scanning methodology known in the art and available to the treating physician. For example, infarct volume can be assessed by the Alberta Stroke Program Early Computed Tomography Score (ASPECTS), this scoring system was applied to images obtained by non-contrast computed tomography (NCCT), post-contrast CT (PCCT), and diffusion-weighted imaging (DWI). DWI stroke volume was semi quantitatively measured with the manually outlined hyperintense lesion. Infarct core volume was calculated with a threshold apparent diffusion coefficient value of 600×10⁻⁶ mm²/s. Intraclass correlation coefficients (ICC) can be estimated to assess inter-reader reliability for ASPECTS scoring and DWI stroke volume.

Inflammatory markers, such as C-reactive Protein (CRP), Interleukin-6 (IL-6), tumor necrosis factor (TNF)-alpha and fibrinogen, are upregulated following neuronal injury such as stroke. It is known that these biomarkers are associated with increased mortality, recurrent vascular risk, and poor functional outcome. These biomarkers can be measured using any known methods. The methods described herein can result in a decrease in these biomarkers compared to an untreated subject.

It is well known that neuronal injuries such as ischemic stroke can cause gray matter and white matter injury. White matter injury is a risk factor for poor neurological outcomes. The majority of damage caused by stroke is located in subcortical regions and white matter can occupy nearly half of the average infarct volume. White matter is exquisitely vulnerable to ischemia and is often injured more severely than gray matter. Clinical symptoms related to white matter injury include cognitive dysfunction, emotional disorders, sensorimotor impairments, as well as urinary incontinence and pain, all of which are closely associated with destruction and remodeling of white matter connectivity. White matter (and gray matter) injury can be noninvasively detected by MRI, which provides a three-dimensional assessment of its morphology, metabolism, and function.

After acute stroke, multiple immune cells can enter the brain parenchyma in an orderly manner. Peripheral immune cells, including myeloid dendritic cells, monocytes/macrophages, and neutrophils appear within 1 day after stroke. Subsequently, small increases of T and B lymphocytes can be detected. Once the brain environment is affected by pathophysiological changes such as stroke, infiltrating cells can differentiate into macrophages which have special functions, including the production of inflammatory mediators and phagocytosis. Infiltrating immune cells mainly differentiate into two phenotypes, the classical pro-inflammatory phenotype (M1) and the alternative anti-inflammatory phenotype (M2). Phenotypic markers of M1 include CD16, CD32, CD86, MHC II, and iNOS, while CD206, Arg1, and Ym1 are identified as M2 phenotype markers. As shown herein, the methods of the invention can reduce the number of CD16/CD32 positive cells in the peri-infarct region.

The Modified Rankin Scale (mRS) is a single item, global outcomes rating scale for subjects post-stroke. It is used to categorize levels of functional independence with reference to pre-stroke activities rather than on observed performance of a specific task.

The conventional method of administration for the mRS is a guided interview process. The assessment is carried out by asking the patient about their activities of daily living, including outdoor activities. Information about the patient's neurological deficits on examination, including aphasia and intellectual deficits, should be obtained. All aspects of the patient's physical, mental performance, and speech should be combined in the choice of a single MRS grade.

A single mRS grade should be assigned based on the following criteria (Dromerick, A. W., Edwards, D. F., Diringer, M. N. (2003). Sensitivity to changes in disability after stroke: A comparison of four scales useful in clinical trials. Journal of Rehabilitation Research and Development, 40(1), 1-8)

TABLE 1
mRS grading

| Rankin Grade | Description |
|---|---|
| 0 | No symptoms |
| 1 | No significant disability despite symptoms: able to |
| | carry out all usual duties and activities |
| 2 | Slight disability: unable to carry out all previous |
| | activities but able to look after own affairs |
| | without assistance |
| 3 | Moderate disability: requiring some help, but |
| | able to walk without assistance |
| 4 | Moderately severe disability: unable to walk |
| | without assistance, and unable to attend to own |
| | bodily needs without assistance |
| 5 | Severe disability: bedridden, incontinent, and |
| | requiring constant nursing care and attention |

The methods disclosed herein can result in an improvement in mRS score.

The National Institutes of Health Stroke Scale, or NIH Stroke Scale (NIHSS), is a tool used to objectively quantify the impairment caused by a stroke. The NIHSS is composed of 11 items, each of which scores a specific ability between a 0 and 4. For each item, a score of 0 typically indicates normal function in that specific ability, while a higher score is indicative of impairment. The individual scores from each item are summed in order to calculate a patient's total NIHSS score. The maximum possible score is 42, with the minimum score being a 0. The methods described herein can result in an improvement in NIHSS score.

TABLE 2
NIHSS grading

| Score | Stroke severity |
|---|---|
| 0 | No stroke symptoms |
| 1-4 | Minor stroke |
| 5-15 | Moderate stroke |
| 16-20 | Moderate to severe stroke |
| 21-42 | Severe stroke |

The items assessed in the NIHSS are as follows.
1a. Level of consciousness. A 3 is scored only if the patient makes no movement (other than reflexive posturing) in response to noxious stimulation.
1b. LOC Questions. The patient is asked the month and his/her age. The answer must be correct. Aphasic and stuporous patients who do not comprehend the questions will score 2. Patients unable to speak because of endotracheal intubation, orotracheal trauma, severe dysarthria from any cause, language barrier or any other problem not secondary to aphasia are given a 1.
2. Best Gaze. Only horizontal eye movements will be tested. Voluntary or reflexive (oculocephalic) eye movements will be scored but caloric testing is not done. If the patient has a conjugate deviation of the eyes that can be overcome by voluntary or reflexive activity, the score will be 1. If a patient has an isolated peripheral nerve paresis (CN III, IV or VI) score a 1. Gaze is testable in all aphasic patients. Patients with ocular trauma, bandages, pre-existing blindness or other disorders of visual acuity or fields should be tested with reflexive movements and a choice made by the investigator. Establishing eye contact and then moving about the patient from side to side will occasionally clarify the presence of a partial gaze palsy.
3. Visual. Visual fields (upper and lower quadrants) are tested by confrontation, using finger counting or visual threat as appropriate. Patient must be encouraged, but if they look at the side of the moving fingers appropriately, this can be scored as normal. If there is unilateral blindness or enucleation, visual fields in the remaining eye are scored. Score 1 only if a clear-cut asymmetry, including quadrantanopia is found. If patient is blind from any cause score 3. Double simultaneous stimulation is performed at this point. If there is extinction patient receives a 1 and the results are also used to answer question 11.
4. Facial Palsy. Ask, or use pantomime to encourage the patient to show teeth or raise eyebrows and close eyes. Score symmetry of grimace in response to noxious stimuli in the poorly responsive or non-comprehending patient. If facial trauma/bandages, orotracheal tube, tape or other physical barrier obscures the face, these should be removed to the extent possible.
5 & 6. Motor Arm and Leg. Each limb is tested in turn, beginning with the non-paretic arm, if known. The limb is placed in the appropriate position: extend the arm (palm down) 90 degrees (if sitting) or 45 degrees (if supine) and the leg 30 degrees (always tested supine). Drift is scored if the arm falls before 10 seconds or the leg before 5 seconds. The aphasic patient is encouraged using urgency in the voice and pantomime but not noxious stimulation. Only in the case of amputation or joint fusion at the shoulder or hip may the score be “9” and the examiner must clearly write the explanation for scoring as a “9”.
7. Limb Ataxia. This item is aimed at finding evidence of a unilateral cerebellar lesion. Test with eyes open. In case of visual defect, ensure testing is done in the intact visual field. The finger-nose-finger and heel-shin tests are performed on both sides, and ataxia is scored only if present out of proportion to weakness. Ataxia is absent in the patient who cannot understand or is paralyzed. Although the use of untestable is discouraged, in the case of amputation, joint fusion or some fractures, the item may be scored “9”, and the examiner must clearly write the explanation for not scoring. In case of blindness, test by touching nose from extended arm position.
8. Sensory. Sensation or grimace to pin prick when tested, or withdrawal from noxious stimulus in the obtunded or aphasic patient. Only sensory loss attributed to stroke is scored as abnormal and the examiner should test as many body areas (arms (not hands), legs, trunk, face] as needed to accurately check for hemisensory loss. A score of 2, “severe or total,” should only be given when a severe or total loss of sensation can be clearly demonstrated. Stuporous and aphasic patients will therefore probably score 1 or 0. The patient with brain stem stroke who has bilateral loss of sensation is scored 2. If the patient does not respond and is quadriplegic, score 2. Patients in coma (item la=3) are arbitrarily given a 2 on this item.
9. Best Language. A great deal of information about comprehension will be obtained during the preceding sections of the examination. The patient is asked to describe what is happening in a picture, to name the items on a naming sheet, and to read from a list of sentences. Have the patient name all items on the naming sheet and read all phrases on two reading sheets. Comprehension is judged from responses here as well as to all of the commands in the preceding general neurological exam. If visual loss interferes with the tests, ask the patient to identify objects placed in the hand, repeat, and produce speech. The intubated patient should be asked to write. The patient in coma (question la=3) will arbitrarily score 3 on this item. The examiner must choose a score in the patient with stupor or limited cooperation but a score of 3 should be used only if the patient is mute and follows no one step commands.
10. Dysarthria. If the patient is thought to be normal, an adequate sample of speech must be obtained by asking the patient to read or repeat words from a list. If the patient has severe aphasia, the clarity of articulation of spontaneous speech can be rated. Only if the patient is intubated or has other physical barrier to producing speech, may the item be scored “9”, and the examiner must clearly write an explanation for not scoring. Do not tell the patient why he/she is being tested.
11. Extinction and inattention. Sufficient information to identify neglect may be obtained during the prior testing. If the patient has a severe visual loss preventing visual double simultaneous stimulation, and the cutaneous stimuli are normal, the score is normal. If the patient has aphasia but does appear to attend to both sides, the score is normal. The presence of visual spatial neglect or anosognosia may also be taken as evidence of abnormality. Since the abnormality is scored only if present, the item is never untestable.

### EXAMPLES

### Example 1: De-Identified and Pooled Umbilical Cord Serum Collected from Cord Blood Bank

Individual umbilical cord blood (UCB) is collected according to the standard American Association of Blood Banks (AABB) protocol. See Badowski M. S., Harris D. T. (2012) Collection, Processing, and Banking of Umbilical Cord Blood Stem Cells for Transplantation and Regenerative Medicine. In: Singh S. (eds) Somatic Stem Cells. Methods in Molecular Biology (Methods and Protocols), vol 879.

An aliquot of umbilical cord blood from each individual is removed for disease and blood group testing.

Following blood group determination and assessment for disease free status, individual samples are pooled and stored frozen at −80° C.

### Example 2: Plasma Preparation

Plasma is prepared from pooled umbilical cord blood by centrifugation.

Human UCB containing citrate phosphate dextrose adenine as an anticoagulant was obtained from the Cord Blood Bank. Only fresh UCB supplied within 24 h postpartum was used in this study.

UCB was centrifuged for 10 min at 1500×g at room temperature and separated into 3 layers; plasma (UCB-PL top layer), a mixture of leucocytes and platelets (middle layer), and erythrocytes (bottom layer).

The UCB-PL layer was carefully collected, aliquoted, and stored as UCF at −80° C. until use.

Acellular plasma (UCF) was prepared by centrifuging the UCB-PL at 3000 g for 20 minutes.

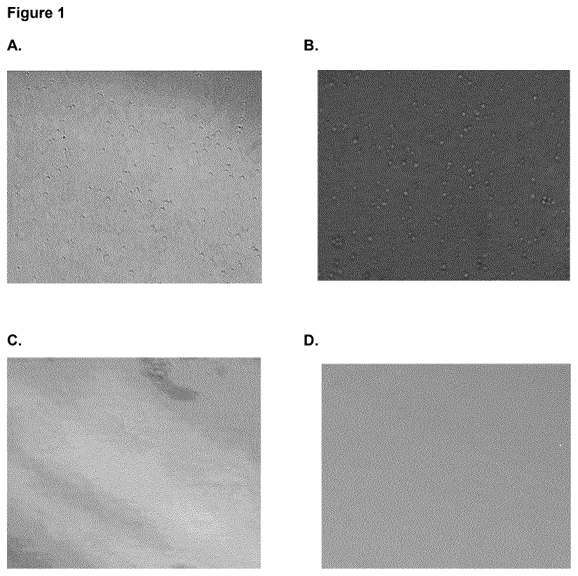

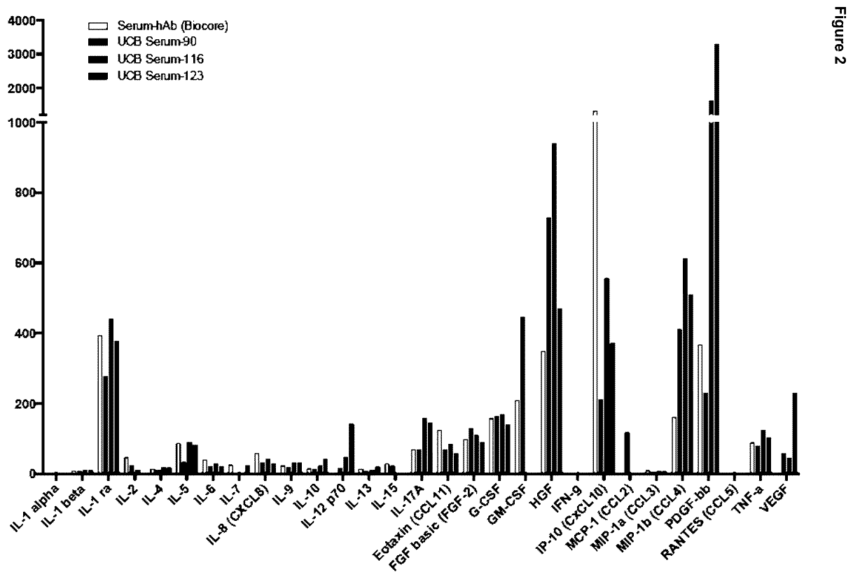

Cell counts of the layers and the acellular plasma were performed using a Guava® Muse® Cell Analyzer (Luminex). The results are presented in Table 3 and in FIG. 1 which shows light microscopy images of each of the middle layer, top layer, acellular plasma and water.

TABLE 3
Cell counts

| Sample | Sample ID 161.5 | Sample ID 61.5 |
|---|---|---|
| Middle | 2.84 × 10⁶ cells/mL (Viable) | 2.83 × 10⁷ cells/mL (Viable) |
| layer | 95% Cell viability | 95% Cell viability |
| | 2.96 × 10⁶ cells/mL (Total) | 2.96 × 10⁷ cells/mL (Total) |
| Top | 6.17 × 10⁴ cells/mL (Viable) | 5.49 × 10⁴ cells/mL (Viable) |
| layer | 98.44% Cell viability | 92.95% Cell viability |
| | 6.27 × 10⁴ cells/mL (Total) | 5.91 × 10⁴ cells/mL (Total) |
| Acellular | 0 cells/mL (Viable) | 0 cells/mL (Viable) |
| plasma | 0% Cell viability | 0% Cell viability |
| | 0 cells/mL (Total) | 0 cells/mL (Total) |
| Water | 0 | 0 |

Cytokine profiling of acellular plasma was performed, and the results are shown in FIG. 2 with normal human (adult) serum as a control.

### Example 2: Exosome Preparation

Cell free frozen cord blood plasma samples (UCF) were thawed and pooled. Exosomes (UCF-E) are prepared using a combination of centrifugation, precipitation, and microfiltration steps. In some cases, a cryoprotectant was added before the exosomes were freeze dried (lyophilized).

The lyophilized exosomes are stable for over 36 months at 4° C. Samples that have been kept at 4° C. or room temperature did not change from the initial assessment. In view of this finding the exosomes remained stable under a wide variety of conditions.

Exosomes were subsequently quantified for overall particle number by NTA (Nanoparticles Tracking Analysis) with Nano Sight LM10. Lyophilization does not affect the stability of purified exosomes (FIG. 2). Exosome Standards lyophilized exosome standards are highly pure, easy to reconstitute and easy to ship and store at 4° C.

TABLE 4
Lyophilized UCF

| | Starting | Concentrated | Final |
|---|---|---|---|
| | material | product | product |
| Product | UCF | UCF Extract | Freeze Dried UCF Extract |
| description | | | |
| Volume/mass | 127 ml | 12 mL | 2.18 g |
| Storage | −20° C. | 2-8° C. | 2-8° C. |
| conditions | | | |
| Physical | Frozen/liquid | Liquid | Friable dry powder |
| properties | | | Theoretical shelf life: 3 |
| | | | years at 4° C. |
| | | | Reconstitute in 12 mL H₂O. |

Samples of the starting material (acellular plasma), concentrated product (exosomes) and final product (freeze dried exosomes) were characterized by Nanoparticle Tracking Analysis (NTA) using a Nanosight NS300 (Malvern Instruments Ltd). NTA analysis provides estimates of EV concentration and size distribution as provided in Table 5.

TABLE 5
NTA Data Summary

| | | | Freeze dried |
|---|---|---|---|
| | | UCF Extract | UCF Extract |
| Fraction | Acellular plasma | (Exosomes) | (Exosomes) |
| Fraction volume | 127.0 | 12.5 | 12.0 |
| (mL) | | | |
| Particle size; mode | 81.8 ± 1.7 | 85.9 ± 2.2 | 92.8 ± 1.5 |
| (nm) | | | |
| Concentration | 2.09 × 10¹¹ ± | 5.07 × 10¹¹ ± | 2.92 × 10¹² ± |
| (particles/mL) | 6.31 × 10⁹ | 9.97 × 10¹⁰ | 2.28 × 10¹¹ |
| Total particles in | 2.65 × 10¹³ ± | 6.34 × 10¹² ± | 3.50 × 10¹³ ± |
| fraction | 8.01 × 10¹¹ | 1.25 × 10¹² | 2.74 × 10¹² |

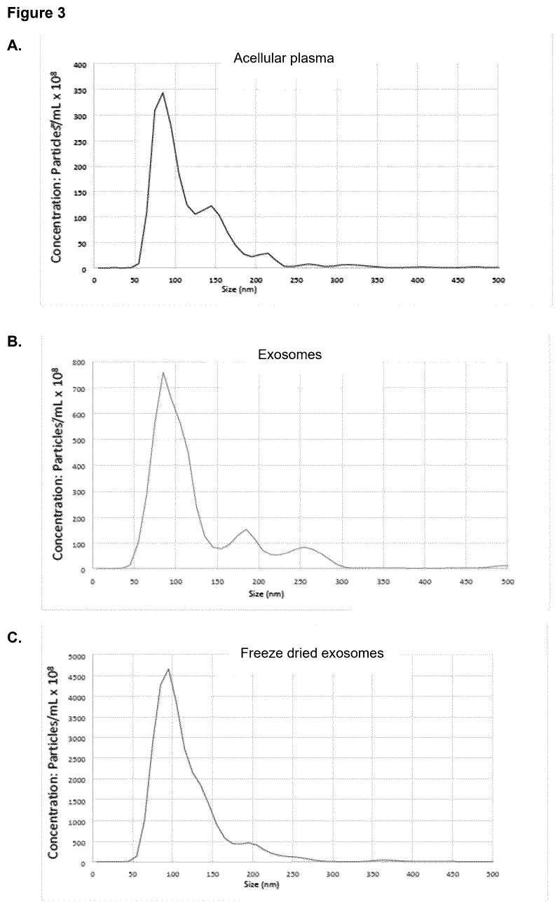

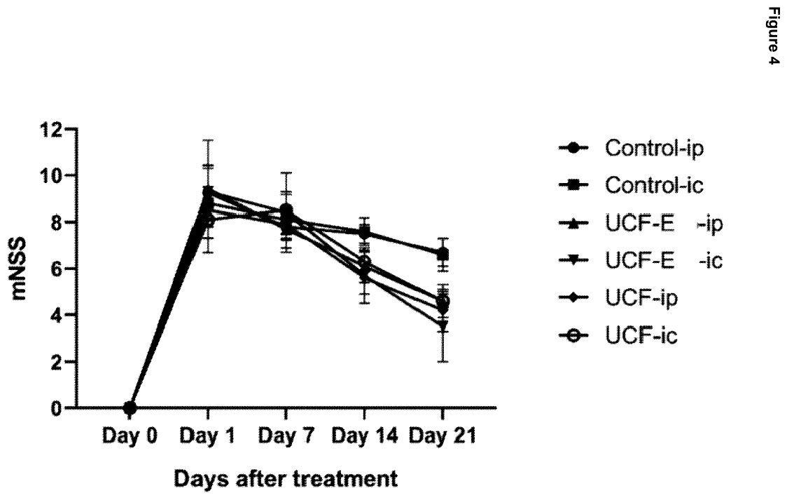

These data are illustrated graphically in FIG. 3.

### Example 3: Lyophilization

The acellular plasma or exosome preparations were first frozen to −80° C. and then rapidly placed in a Labconco Freezone 12 Litre Console Freeze Dryer with the condenser maintained at −35° C. under 100 mTorr (a vacuum strong enough to rapidly freeze water) for 24 h before resealing the vials. There was no heat applied, and no organic solvents or salts were added, and the samples remained frozen under strong vacuum during the drying by means of a −45° C. condenser and thus were lyophilized.

The following protocol was used to lyophilize the acellular plasma or exosome preparations.

TABLE 6
Thermal Treatment Steps

| Step | Temp | Time | Ramp/Hold |
|---|---|---|---|
| 1 | −45 | 480 | H |
| 2 | −99 | 0 | H |
| 3 | −99 | 0 | H |
| 4 | −99 | 0 | H |
| 5 | −99 | 0 | H |
| 6 | −99 | 0 | H |
| 7 | −99 | 0 | H |
| 8 | −99 | 0 | H |
| 9 | −99 | 0 | H |
| 10 | −99 | 0 | H |
| 11 | −99 | 0 | H |
| 12 | −99 | 0 | H |

Freeze temperature of −40° C., additional freeze time of 20 minutes. Condenser setpoint of −40° C. and a vacuum setpoint of 150 mTorr.

TABLE 7
Primary Drying Steps

| Step | Temp (° C.) | Time (sec) | Vac (mTorr) | Ramp/Hold |
|---|---|---|---|---|
| 1 | −35 | 100 | 100 | H |
| 2 | −30 | 570 | 100 | H |
| 3 | −35 | 390 | 100 | H |
| 4 | −10 | 300 | 100 | R |
| 5 | −10 | 240 | 100 | H |
| 6 | 0 | 100 | 100 | R |
| 7 | 0 | 630 | 100 | H |
| 8 | −99 | 0 | 0 | H |
| 9 | −99 | 0 | 0 | H |
| 10 | −99 | 0 | 0 | H |
| 11 | −99 | 0 | 0 | H |
| 12 | −99 | 0 | 0 | H |
| 13 | −99 | 0 | 0 | H |
| 14 | −99 | 0 | 0 | H |
| 15 | −99 | 0 | 0 | H |
| 16 | −99 | 0 | 0 | H |
| Post Heat | 20 | 1200 | 70 | |

Secondary temperature was 30° C.

### Example 4: Reconstitution Protocol

285 mg lyophilized acellular plasma with exosomes (UCF) were reconstituted with 5 mL of water for injection. The powder was completely dissolved and had the appearance of serum (a straw (yellow) color). The reconstituted acellular plasma with exosomes was filtered through a 0.2 μm filter. The sterile filtered acellular plasma with exosomes (UCF) can be used immediately or stored at 2-8° C. for two weeks.

86 mg lyophilized exosomes (UCF-E) were reconstituted with modified 0.9% Saline (85% Saline)−0.85× solution of 0.9% saline with water for injection (8.5 mL of 0.9% Saline:1.5 mL of water for injection). Prior to reconstitution, the 85% saline is warmed to 37° C. 5 mL of warmed (37° C.) saline was added to completely dissolve the contents. In some cases, incubation at 37° C., with or without occasional vortexing, may be required. The powder was completely dissolved and had the appearance of an offwhite colloid liquid. The reconstituted exosome preparation was a uniform hazy solution and was filtered through a 0.2 μm filter. The sterile filtered exosomes (UCF-E) can be used immediately or stored at 2-8° C. for two weeks.

### Example 5: Animal Study of Acellular Plasma and Exosomes in the Treatment of Middle Cerebral Artery Infarction in Rats

Adult male Sprague-Dawley rats (250-300 g, n=84) were purchased from the Center for Experimental Animal Research of the Chinese Academy of Medical Sciences (Beijing, China). The housing conditions included a 12-hour light/dark cycle, controlled temperature and humidity, and free access to food and water. Rats were randomly assigned to two cohorts and subjected to transient focal cerebral ischemia and reperfusion followed by treatment.

Cerebral ischemia was induced by a transient intraluminal vascular occlusion method using the intraluminal filament technique. In brief, under halothane (Lunan Pharmaceutical, Jinan, China) anaesthesia, the right common carotid artery (CCA), external carotid artery (ECA), and internal carotid artery (ICA) were exposed. The ECA was cut with micro-scissors, and a monofilament nylon thread 0.25 mm in diameter (Sunbio Biotech, Beijing, China) was inserted into the stump of the ECA. The nylon thread was placed into the ICA until resistance was encountered (1.8-2.1 cm from the bifurcation of the carotid artery). After a period of occlusion (120 min), the nylon thread was withdrawn. A knot with a silk suture was tied on the external carotid artery at bifurcation to stop bleeding. The skin was sutured to close the neck incision. Rats from both the cohorts subjected to middle cerebral artery occlusion (MCAO) procedure were administered the recommended doses of buprenorphine, and cefazolin to mitigate pain and prevent infection respectively. Immunosuppressants were not administered.

The modified neurological severity scores (mNSSs) test was then performed after the operation. Rats subjected to transient MCAO with mNSSs around 10-12 were randomly assigned to four groups (A-D groups, for group A, B and C, the animals were subdivided into intracranial and intraperitoneal subgroups) as shown in Table 8.

Reconstituted acellular plasma with exosomes (UCF) or exosomes only (UCF-E as described above) were administered to rats having permeant MCAO (pMCAO) in the early subacute stage which corresponds to 3 days after pMCAO in rats (7 days after an infarct in humans). The treatment regime is set out in Table 8.

At post-ischemic day (PID) 3, MCAO animals were randomly divided into 8 experimental groups. For the intraperitoneal treatment group, acellular plasma (which contains exosomes, UCF) (0.5 mL), exosome only (UCF-E) (0.5 mL) or saline (0.5 mL) were administered into the abdominal cavity over 5 min. For the intracerebral treatment group, three holes (A for 30 μl, B for 30 μl, C for 40 μl—see Table 9) were drilled on the right side of the rat skull (Table 9) and acellular plasma (UCF) (0.1 mL), exosome only (UCF-E) (0.1 mL) or saline (0.1 mL) were stereotactically injected. All the targets were selected around the marginal area of the infarct.

TABLE 8
Treatment regime for rats

| | | | Dose (mL) | | |
|---|---|---|---|---|---|
| | | | Reconstituted | | |
| | | | acellular | Reconstituted | |
| Treatment | Route | Rats | plasma* | exosomes | Saline |
| A: Acellular | Intracranial | 12 | 0.1 | — | — |
| plasma with | Intraperitoneal | 12 | 0.5 | — | — |
| exosomes (UCF)* | | | | | |
| B: Exosomes only | Intracranial | 12 | — | 0.1 | — |
| (UCF-E) | Intraperitoneal | 12 | — | 0.5 | — |
| C: Excipient only | Intracranial | 12 | — | — | 0.1 |
| (isotonic saline) | Intraperitoneal | 12 | — | — | 0.5 |
| D: Control - no | Intracranial | 12 | — | — | — |
| injection | Intraperitoneal | 12 | — | — | — |

*Contains exosomes

TABLE 9
Hole coordinates

| R(mm) AP | (mm) | DV(mm) | V(μL) |
|---|---|---|---|
| A3 | +2 | 3 | 30 |
| B3 | +0.5 | 5 | 30 |
| C3 | −0.5 | 3 | 40 |

R: right lateral to the midline;
AP: anterior to the bregma;
DV: below the dura;
V: volume

### 5.1 Functional Evaluation

Modified Neurological Severity Scores (mNSSs) Test

The mNSS test is the standard and globally accepted method to assess the severity of post-stroke injury and recovery. This test is a composite of motor, sensory, reflex and balance tests. A cumulative score from all the tests determines the severity of the injury. An mNSS score of 13-18 indicates severe injury, 7-12 indicates moderate injury and 1-6 means mild injury. The mNSS test was performed on rats of both the cohorts before ischemia and at regular intervals (1 d, 7 d, 14 d and 21 d) until 21 days reperfusion.

### Cylinder Test

The cylinder test was used as a measure of forelimb asymmetry by observing the rat's movements over 3-minute intervals in a transparent, 18-cm-wide, 30-cm high transparent Plexiglas cylinder, with sufficient diameter to permit movement, and ability too promote rearing and wall exploration. A mirror behind the cylinder made it possible to observe and record forelimb movements when the rat was facing away from the examiner. After an episode of rearing and wall exploration, a landing was scored for the first limb to contact the ground or for both limbs if they made simultaneous contact. Percent use scores were calculated for both the unimpaired and impaired limb, relative to the total number of movements. Percentage use of the impaired limb was subtracted from percentage use of the unimpaired limb to yield an overall limb bias score. Wall exploration and landing movements were analyzed separately. Rats were tested 1-4 weeks after focal ischemia following transplantation.

### Limb-Placing Test

Limb-placing test that assess the sensorimotor integration of the forelimb and the hind limb by checking responses to tactile and proprioceptive stimulation was performed according to the method as described by De Ryck et al (Brain Research. 1992; 573(1):44-60), and modified by Puurunen K et al. (Neuropharmacology. 2001; 40(4): 597-606, and Exp Neurol. 2001; 167(2):348-55). Both sides of the body were tested 24 hour after focal ischemia. The test consisted of seven limb-placing tasks, which were scored by a tester blind to the treatment groups. The following scores were used to detect impairment of the hindlimb and the forelimb: 0, no placing; 1, incomplete or delayed placing; 2, complete, immediate placing. Average placing scores (forelimb and hindlimb combined) for each week of testing were calculated for each rat.

### TTC Staining

Rats treated with plasma (UCF) and exosomes (UCF-E) were deeply anesthetized with pentobarbital and decapitated. Brains were removed, placed in adult rat brain matrix (Kent Scientific Corporation, USA), frozen at approximately −70° C. for about 8-10 min, sliced into 2 mm thick coronal sections. Coronal brain sections were incubated in 2% 2, 3,5-triphenyl tetrazolium chloride (TTC) solution for 30-45 min in the dark followed by the capture of images using the Olympus SZX12 research stereomicroscope. The areas of ischemic and non-ischemic regions of the ipsilateral hemisphere as well as the contralateral hemisphere of each section were traced and measured using Image J analysis software (NIH). Total volumes of each region of rat brains were calculated. The percent infarct size in each rat was calculated by using the formula, infarct size (%)={(volume of the contralateral hemisphere)−(volume of the non-ischemic ipsilateral hemisphere)}×100/volume of the contralateral hemisphere. This formula accounts for the possible interference of brain edema on infarct volume. Ipsilateral hemisphere swelling in each rat was calculated by using the formula, swelling (%)={(volume of the ipsilateral hemisphere)-volume of the contralateral hemisphere)}×100/volume of the contralateral hemisphere.

### MRI Scanning

Seven days after the surgery or treatment, rats were anesthetized with isoflurane 1.5%-2% for MRI. During the MRI procedure, the rats were kept at 37° C. and an MRI compatible respiration sensor was used to control the animals. The MRI experiments consisted of three dimensional (3D) T2 weighted image (T2WI), a series of diffusion-weighted imaging (DWI) to calculate the apparent diffusion coefficient (ADC) maps and a series of perfusion-weighted imaging to calculate cerebral blood flow maps. The perfusion experiments were carried out using arterial spin labeling techniques without contrast agent injection. All MRI experiments were performed on a BIOSPEC BMT 47/40 (Bruker, Ettlingen, Germany) spectrometer operating a 4.7 T, equipped with an 11.2-cm actively shielded gradient system, capable of 200 mT/m gradient strength and 80 s of rise time. A 7-cm birdcage radiofrequency coil was used for transmission and reception. T2WI, DWI, and perfusion-weighted imaging image analysis was performed using ParaVision 3.0.1 (Bruker, Ettlingen, Germany). The ADC maps were calculated from the DWI series using the image sequence analysis tool of the ParaVision package. The cerebral blood flow maps were calculated from the arterial spin labeling images. This calculation was carried out using Matlab 7.3 (MathWorks, Inc., Natick, MA), the data were shown in % of brain volume affected.

### Neuroprotection

To assess gray and white matter damage, coronal paraffin sections (6 μm) were incubated with MAP2 (Sigma Aldrich) or mouse-anti-myelin basic protein (MBP, Sternberger Monoclonals, Lutherville, MD), and binding was visualized with a Vectastain ABC kit (Vector Laboratories, Burlingame, CA). Brain damage was analyzed at a location equivalent to −1.58 mm from bregma in adult mice by outlining both hemispheres on full-section images using ImageJ software (Rasband WS, ImageJ, NIH, Bethesda, Maryland; http://rsb.info.nih.gov/ij/, 1997-2009). Ipsilateral MAP2 and MBP area loss were calculated as described.

### Cell Death

Cell death was detected in the peri-infarct zone by TUNEL staining (TdT-FragEL DNA Fragmentation Detection Kit, Oncogene Research Products). Cell counts were performed on one slice from each rat (n=10 per group), which was taken at 1.6 mm posterior to the bregma. The number of positive cells was counted in a minimum of 10 different microscopic fields based on their nuclear morphology and dark color using a 40× objective and image analysis software (Image-Pro Plus 4.1, Media Cybernetics).

### Neuroinflammation

Microglia activation was evaluated by measuring their transformation shape. Twenty-five microglial cells were randomly selected in a 300-m wide area at the peri-infarct zone per section, in 6 coronal sections spaced 240 m apart (150 microglial cells per animal) (n=4 per group) 1 and 7 days after treatment. The cell nucleus was placed in the center of a grid of 10 concentric 5 m apart and the number of intersections between cytoplasmic processes and grid lines was registered using 100× oil immersion lens.

### Glial Scar

GFAP expression in the border of infarct zone was quantified by measuring integrated optical density and area fraction with ImageJ software (National Institute of Mental Health, Bethesda, MA).

### Statistical Analysis

All data are expressed as means±SEM. Functional outcomes measured with cylinder rearing test were analyzed using two-way ANOVA with Fisher's least significant difference post-tests. P<0.05 was considered statistically significant. Histological measures were analyzed using one-way ANOVA with Bonferroni post-tests. P<0.05 was considered statistically significant.

### 5.2 Results

### Neurological Outcome

mNSS after MCAO are shown in FIG. 4. Neurological functions in all six groups gradually recovered over time (see FIG. 4). mNSS was significantly higher in animals treated with saline group than that in animals treated with UCF (acellular plasma with exosomes) and UCF-E (exosomes only) at 14 d (control-ip vs. UCF-ip, UCF-ic, UCF-E-ip and UCF-E-ip, P<0.05, or control-ic vs. UCF-ip, UCF-ic, UCF-E-ip and UCF-E-ic, P<0.05) and 21 d (control-ip vs. UCF-ip, UCF-ic, and UCF-E-ip P<0.05, or control-ic vs. UCF-ip, UCF-ic and UCF-E-ip P<0.05; control-ic vs. UCF-E-ic, control-ip vs. UCF-E-ic, P<0.01) after MCAO (P<0.05). The higher the mNSS, the higher the severity of neurological injury. The score in the UCF-E-ic (3.5±1.5) is lower than the other three treatment groups (4.5±0.7 in UCF-ic group, 4.2±0.9 in UCF-ip group, 4.6±0.3 in UCF-E-ip group) at 21 d following treatment. However, no significant difference was found among rats treated with UCF and UCF-E intracranially or intraperitoneally.

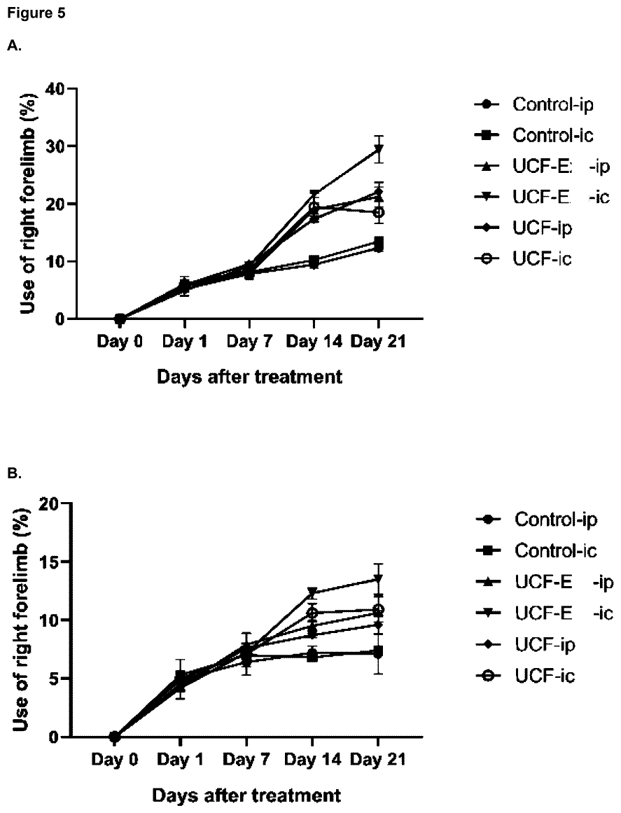

Using the cylinder and the limb placing score, rats that had received UCF (acellular plasma with exosomes) and UCF-E (exosomes only) injection intracranially and intraperitoneally on day 14 and 21 showed better functional outcome than saline controls (FIGS. 5A-B). Noteworthy, rats that had received UCF-E-ic on day 14 and 21 post stroke also significantly performed better in the behavioural tests than rats that received UCF-ic, UCF-ip, or UCF-E-ip (FIGS. 5A-B). However, better test scores were not immediately evident at the beginning of the behavioural tests.

### Evaluation of Infarction Volume

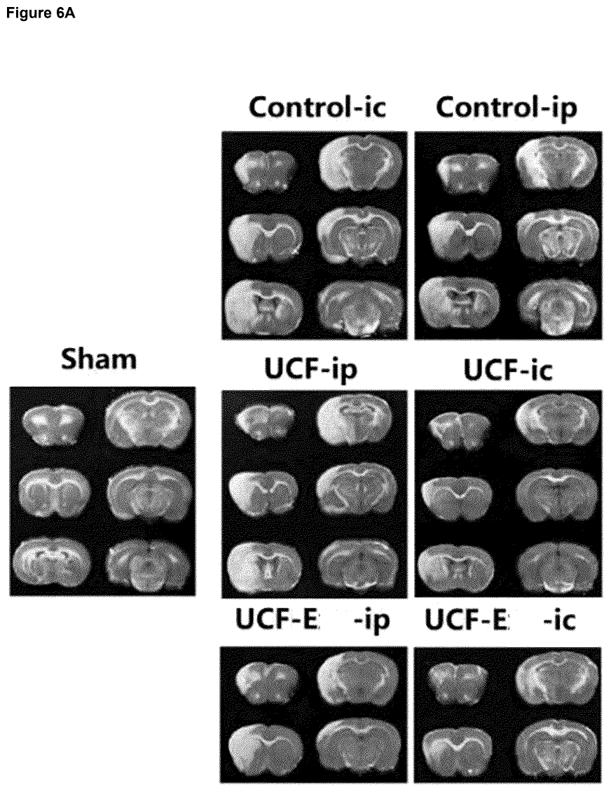

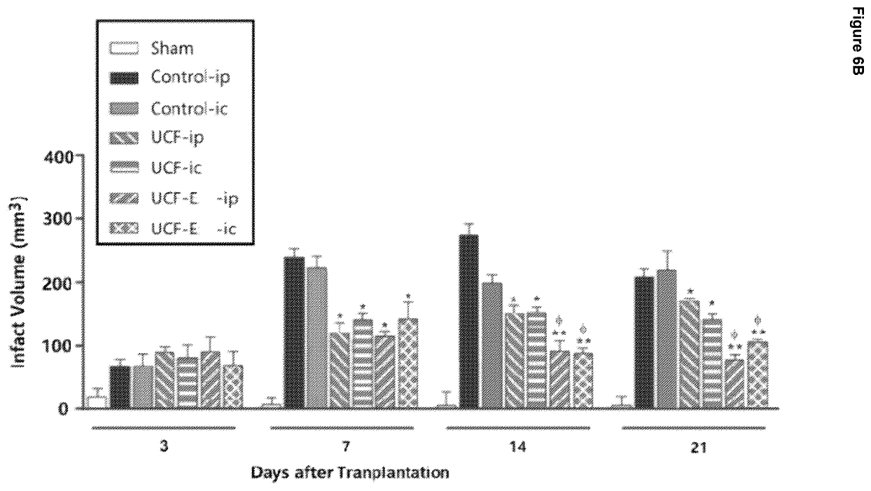

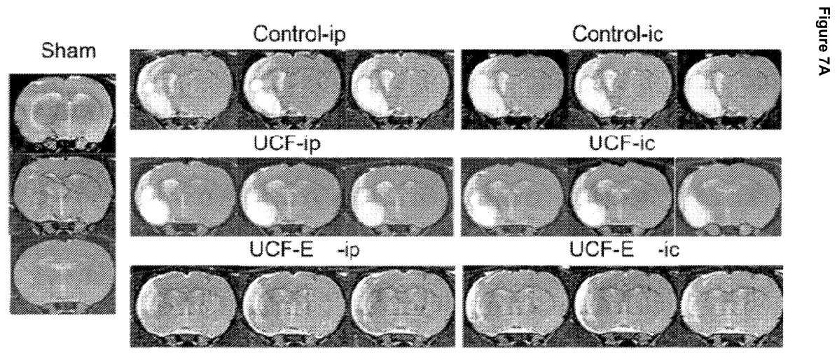

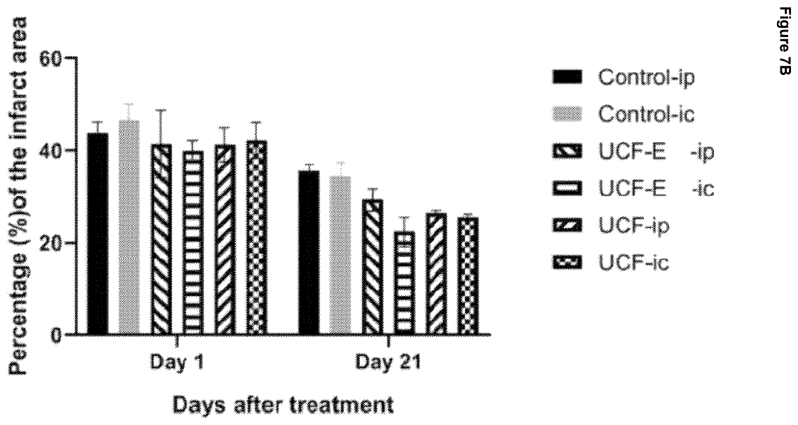

At 14 and 21 days after treatment, the infarct volume in UCF-ic and UCF-E-ip groups were significantly lower than those of the saline-ic and saline-ip group (UCF-ic vs. Control-ic, UCF-ip vs. Control-ic, UCF-ic vs. Control-ip, UCF-ip vs. Control-ip, **P<0.05; UCF-E-ic vs. Control-ic, UCF-E-ip vs. Control-ic, **P<0.01, FIG. 6). However, no significant difference was found among rats treated with UCF and UCF-E intracranially or at 7 days after treatment. Rats subjected to intraperitoneally UCF or UCF-E infusion showed a larger lesion size compared with the intracranially UCF or UCF-E injection groups at 14 and 21 days after injection.

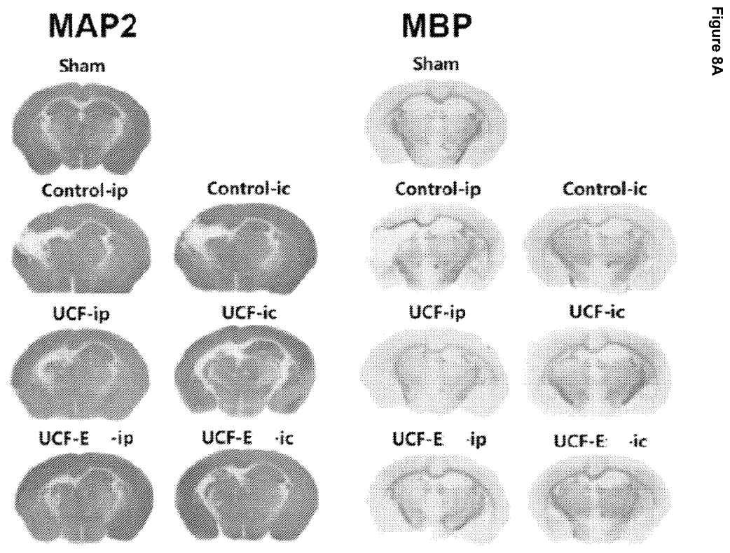

Lesion sizes at 1 d and 14 d after the experimental procedure were evaluated on MRI (FIG. 7). The data of MRI scanning were similar to those of the TTC staining.

Rats subjected to intraperitoneally UCF or UCF-E infusion showed a larger infarct rate compared with the intracranially UCF or UCF-E injection groups at 14 and 21 days after injection.

### Neuroprotection

Effect of treatment with UCF and UCF-E on lesion size was determined at 21 days after cerebral ischemia by analyzing the loss of ipsilateral gray (mouse-anti-microtubule-associated protein (MAP2) and white (mouse-anti-myelin basic protein (MBP) matter.

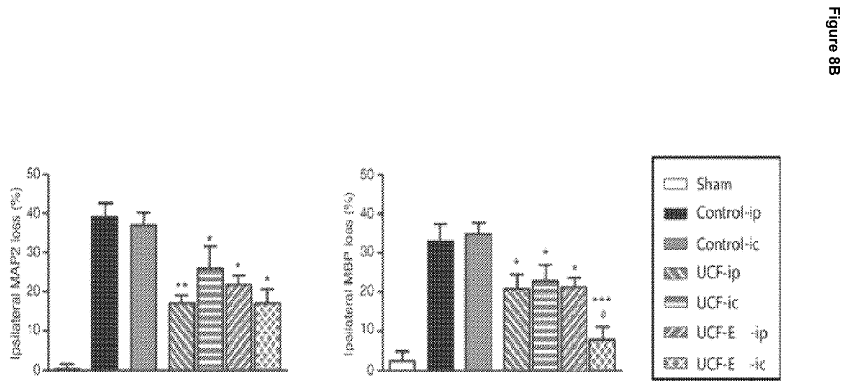

In saline-treated mice, ipsilateral MAP2 area loss was 39.13±3.5% and 37.86±7.1%, and the value did not differ between the two groups (ip vs ic). Treatment with UCF-E (ic and ip) or UCF (ip) significantly reduced lesion volume when compared with saline treatment (as shown in FIG. 8, ***P<0.001, **P<0.01, *P<0.05, respectively). There were no significant differences with the ipsilateral MAP2 area loss among the four treatment groups.

In saline-treated rats, ipsilateral MBP area loss was 35.26±4.2% and 36.91±3.3% (FIG. 8), and the value did not differ between the two groups (ip vs ic). Treatment with UCF-E (ic and ip) or UCF (ic and ip) significantly reduced lesion volume when compared with saline treatment (as shown in FIG. 8, ***P<0.001, **P<0.01, *P<0.05, respectively). There were no significant differences with the ipsilateral MAP2 area loss among group UCF-ip, UCF-ic or UCF-E-ip. However, ipsilateral MAP2 area loss in UCF-E-ic treated rat was significantly lower than in UCF-ip, UCF-ic or UCF-E-ip treated rats at 21 days after treatment.

### TUNEL Staining

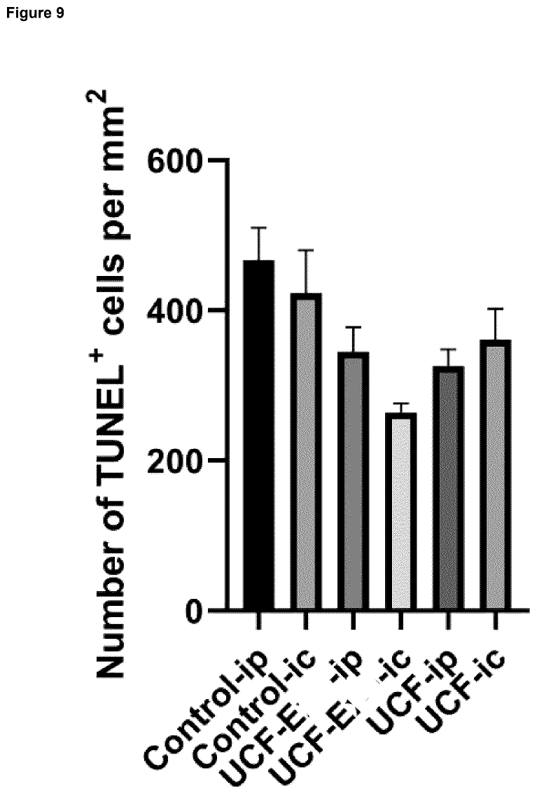

TUNEL staining was used to determine the number of apoptotic cells in the para-infarct areas 14 days after treatment (FIG. 9). The mean numbers of TUNEL positive cells per mm² were as follows: 467±43.2 (control-ip), 8.25±2.693 (sham), 20.45±10.308 (ischemia), 20.15±9.67 (vehicle), and 10.0±3.974 (treatment). The four treatment groups had fewer apoptotic cells than the two control groups. A significant difference existed in the number of TUNEL positive cells between the control and treatment groups (as shown in FIG. 9). The number of TUNEL positive cells in UCF-E-ic treated group was significantly lower than in the UCF-ip, UCF-ic or UCF-E-ip treated group at 14 days after treatment.

### Glial Scar

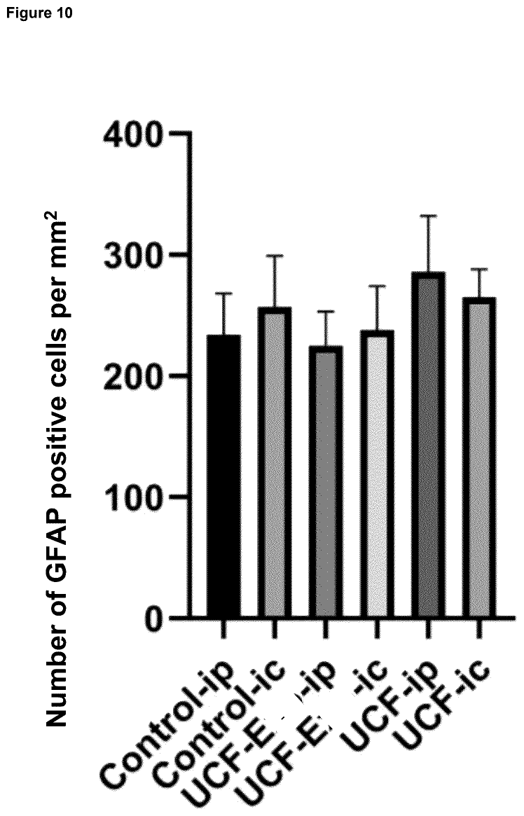

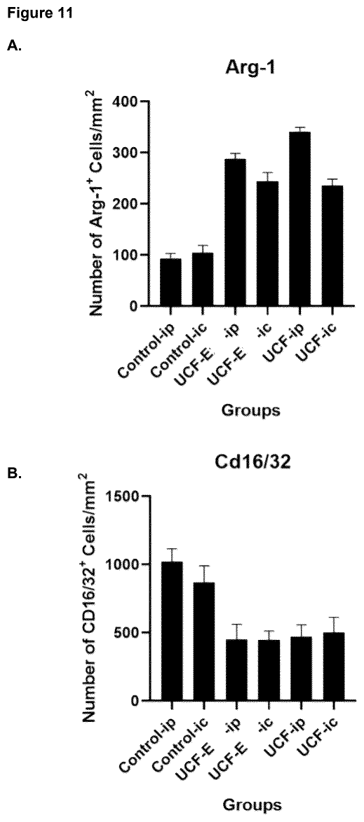

There were no significant differences in GFAP positive cells among different groups at 21 days after treatment (FIG. 10).

### Macrophage Phenotype

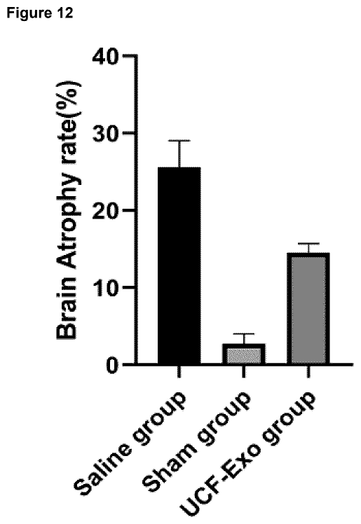

To investigate whether the administration of UCF-E or UCF (ip or ic) may influence the macrophage phenotype, the expression profiles of macrophages were evaluated by immunofluorescence staining with CD16/32 and arginase-1 (Arg-1). Fourteen days post-injury, higher numbers of arginase-1-positive cells were found in the four treatment groups compared to the two control groups (all P<0.01). The number of Arg-1 positive cells in UCF-ip treated group was significantly higher than in the UCF-ic, UCF-E-ic or UCF-E-ip treated group at 14 days after treatment (all P<0.01). Furthermore, the number of CD16/32-positive cells was significantly reduced in animals that received UCF-ic, UCF-ip UCF-E-ic or UCF-E-ip injections compared to animals in the two saline control groups. There were no significant differences observed among the four treatment groups. See FIG. 11.

### 5.3 Conclusions

Both UCF (acellular plasma with exosomes) and UCF-E (exosomes only) are safe for intraperitoneal or intracranial injection, and no injection-related complications or deaths occurred.

Intraperitoneal or intracranial injection of UCF and UCF-E can promote functional recovery after stroke in rats. Although there is no statistically significant difference between them (this may be related to the relatively small number of experimental rats and the short observation period), UCF-E intracranial injection has a higher score in absolute figure.

The repair mechanism of UCF and UCF-E may be related to neuroprotection, which can be seen from the reduction of damage area, staining of neuron-related markers and reduction of apoptosis.

UCF and UCF-E have little effect on gliosis, so they have little effect on reducing glial scar formation in the chronic stage.

UCF and UCF-E have strong regulatory effects on the local immune inflammatory response of the central nervous system.

### 6. Human Study

A small study was conducted in six volunteers to observe the safety and initial effectiveness of UCF and UCF-E in humans. The study passed the ethical review of the 7th Medical Center of the PLA General Hospital (number 2021-1368-6) and was conducted strictly in accordance with the ethical review opinions and experimental protocol of the 7th Medical Center of the PLA General Hospital.

The inclusion criteria for the study were as follows:
1. Stroke duration of at least 3 months but no more than 24 months accompanied by motor nerve dysfunction.
2. Men and women aged 30-65.
3. Women must have a negative serological pregnancy test result and be using appropriate contraception or be infertile. (Menopausal for at least two years or have undergone hysterectomy, oophorectomy, or sterilization).
4. In the subcortical region supplied by the middle cerebral artery (MCA) or lenticulostriate artery, there is a clear history of complete ischemic stroke involving or not involving cerebral cortex ischemic injury which can be found by head MRI.
5. Modified Ranking score: 2, 3 or 4.
6. The FMMS score is no higher than 55.
7. The NIHSS score fluctuated between positive and negative 4 when evaluated twice at least 3 weeks before surgery.
8. Be able and willing to complete all follow-up visits.
9. Be able and willing to carry out late physical therapy and rehabilitation therapy.

The exclusion criteria for the study were as follows:
1. Any psychological or psychiatric illness that may interfere with the study.
2. Patients who have a history of more than one symptomatic stroke except for Transient Ischemic Attacks (TIAs).
3. Patients who have a history of other serious neurological diseases or injuries.
4. The cerebral infarction area was bigger than 8 cm in any measurement.
5. A history of myocardial infarction within 3 months.
6. Patients who have a history of epilepsy or are currently taking antiepileptic drugs.
7. Patients who participated in any other clinical studies of drugs or medical devices within 30 days.
8. In addition to blood transfusion, patients have received other cell transfusions.
9. Any concomitant disease or condition such as:
   a. Coagulation dysfunction and preoperative examination INR >1.4
   b. Panel Reactive Antibodies (PRA)>20% during initial screening
   c. Preoperative active infection
   d. Hypotension requiring hypertensive therapy
   e. Skin damage in the surgical area
   f. Malignant tumour or previous history of malignancy
   g. Primary or secondary immune deficiency
   h. Continuous MRI artifacts and no clear MRI images can be obtained preoperatively or postoperatively, or MRI examination cannot be performed.
   i. Serum creatinine >115 μmol/L, liver function (aspartate aminotransferase/Alt, total bilirubin, alkaline phosphatase) >2 times the upper limit of normal, haematocrit (%)/(g/L) haemoglobin <30/10, the total number of white blood cells <4×109/L, the platelet count <100×109/L), uncontrolled hypertension, systolic blood pressure >180 MMHG or diastolic blood pressure >100 MMHG or uncontrolled diabetes (hba1c>8%), occult blood test found that the digestive tract haemorrhage, positive tuberculosis (TB skin test: PPD/tuberculin test), hepatitis B or C, or human HIV positive.
10. Any of the following conditions including:
   a. Recent drug or alcohol abuse
   b. An unstable state of health
   c. Unstable mental illness including psychosis and untreated major depression.
11. Any situation considered by the investigator or attending physician that may affect the participation in the clinical study or may bring harm to the subjects.
12. Any situation that the surgeon considers likely to cause postoperative complications.
13. Patients are known to be allergic to tacrolimus or methylprednisolone.
14. Patients are unable or unwilling to participate in physical and/or professional treatment as scheduled, and unable or unwilling to return to the hospital for follow-up as scheduled.
15. Failure to provide informed consent as required by the screening program.
16. Take antiplatelet drugs within 2 weeks before operation.

The evaluation criteria were as follows:

### Safety Assessment

Safety was assessed by adverse events, clinical tests, vital signs, and physical examination.

Follow-up endpoints associated with intravenous injection were reported including 30-day survival, incision infection, epilepsy, meningitis, cerebral hematoma, new ischemic stroke, exacerbation, and secondary surgery.

Other safety endpoints including the frequency of complications associated with:
a. Related to immunosuppressive therapy, including but not limited to nephrotoxicity (creatinine ≥177 mol/L[2 mg/dL], or ≥2 baseline creatinine) or renal failure (requiring renal replacement therapy).
b. Hyperkalaemia or hypomagnesemia due to renal insufficiency.
c. Opportunistic infections (bacterial, fungal or viral including cytomegalovirus).
d. Central nervous system neurotoxicity caused by tacrolimus (including posterior leukoencephalopathy syndrome, epilepsy, focal neurological dysfunction, tremor and headache).
e. New-onset glucose intolerance, hyperglycaemia or diabetes.
f. Gastrointestinal disorders (including anorexia, nausea, vomiting, diarrhea and abdominal discomfort).
g. Malignant neoplasms (squamous cell carcinoma or malignant lymphoproliferative disease) and malignant hypertension.
h. New complications related to stroke or neurological function including increased motor dysfunction (motor function score results), increased sensory dysfunction (neurological examination), or poor cognitive test results (using a neuropsychological kit).
i. Other complications such as urinary tract infections, deep vein thrombosis (DVT), pulmonary embolism, gastric ulcers, bedsores, respiratory infections (pneumonia or tracheobronchitis), death within 30 days of surgery and prolonged hospital stays.

### Evaluation of Efficacy

The primary efficacy measures were:
a. Adverse events.
b. Serious adverse events (SAE).
c. Regular brain MRI scans (plain and cerebral perfusion imaging).
d. Serology and hematology.
e. Clinical assessments of brain injury including National Institutes of Health Stroke Scale (NIHSS), Modified Rankin Score (MRS), Fugl-Meyer Stroke Scale and Cognitive Kits.
f. Detection of fluorodeoxyglucose uptake by positron emission tomography (FDG-PET).

The secondary efficacy measures were:
a. Graft survival at the graft site was determined by MRI (with or without the use of gadolinium-containing contrast agents) and autopsy.
b. The effectiveness of immunosuppressive therapy was evaluated by detecting donor-specific HLA antibodies.

### Patient Information

Three patients meeting the inclusion criteria were selected and provided informed consent. The demographic and clinical information for the patents is set out in Table 10 and the results after administration of UCF-E are in Table 11.

200 μL UCF-E was administered intrathecally to one patient and 400 μL UCF-E was administered intrathecally to the other two patients.

TABLE 10
Patient information

| | | |
|---|---|---|
| Patient 1 | Patient 2 | Patient 3 |

Basic information

| | | | |
|---|---|---|---|
| Age | 54 | 57 | 46 |
| Gender | M | F | M |
| Hypertension history | Y | Y | Y |
| Diabetes history | N | Y | N |
| Hyperlipidemia | N | Y | N |
| history | | | |
| Antiplatelet and | Y | N | N |
| Anticoagulant Drugs | | | |

Clinical information

| | | | |
|---|---|---|---|
| Location of | Left basal | Left basal | Right basal |
| hemorrhage | ganglia | ganglia | ganglia |
| Volume of | 48 | 64 | 67 |
| hemorrhage (mm³) | | | |
| mRS | 3 | 4 | 4 |
| NIHSS | 4 | 8 | 5 |

mRS: modified Rankin scale.
NIHSS: NIH (National Institutes of Health) stroke scale

TABLE 11
Assessment after injections of UCF-E

| | Patient 1 | Patient 2 | Patient 3 |
|---|---|---|---|
| Time from onset of | 8 | 7 | 8 |
| stroke to injection | | | |
| (days) | | | |
| Time of assessment | 26 | 18 | 13 |
| after injections | | | |
| (days) | | | |
| Symptoms and | Severe aphasia | Significant | Significant |
| signs | resolved to | improvement in | improvement in |
| | moderate aphasia; | mental state; and | mental state; and |
| | Salivation improved | Moderate | Moderate |
| | significantly; | improvement of right | improvement of left |
| | Moderate | upper and lower | leg and lower limb |
| | improvement of right | limb dystonia. | dystonia. |
| | leg dystonia; and | | |
| | Significant | | |
| | improvement in | | |
| | mental state. | | |
| mRS | 3 | 4 | 4 |
| NIHSS | 4 | 8 | 5 |

mRS: modified Rankin scale.
NIHSS: NIH (National Institutes of Health) stroke scale

### 7. Human Study in Progress

The above patients are part of a larger study which is designed as follows and is in progress.

TABLE 12
UCF-E Intrathecal injections

| | Dose per | Total | |
|---|---|---|---|
| ID grouping | injections | injections | Frequency |
| A (n = 2) | 200 μL | 3 | 30 days/1 injection |
| B (n = 2) | 400 μL | 3 | 30 days/1 injection |
| C (n = 20) | 800 μL | 3 | 30 days/1 injection |

TABLE 13
UCF-E Intravenous injection

| | Dose per | Total | |
|---|---|---|---|
| ID grouping | injections | injections | Frequency |
| A (n = 2) | 10 mL | 3 | 30 days/1 injection |
| B (n = 2) | 20 mL | 3 | 30 days/1 injection |
| C (n = 20) | 30 mL | 3 | 30 days/1 injection |

One patient who received 3 intrathecal injections (total dose volume is 1.5 ml) of exosomes (UCF-E) at 4 months after hemorrhagic stroke has regained the ability to walk unaided for short distances. Formal assessment of the patient after exosome injections (UCF-E) has been delayed due to Covid restriction. Video data collected and were taken by the family.

TABLE 14
UCF Intrathecal injections

| | Dose per | Total | |
|---|---|---|---|
| ID grouping | injections | injections | Frequency |
| A (n = 2) | 1000 μL | 3 | 30 days/1 injection |
| B (n = 2) | 2000 μL | 3 | 30 days/1 injection |
| C (n = 20) | 3000 μL | 3 | 30 days/1 injection |

TABLE 15
UCF Intravenous injections

| | Dose per | Total | |
|---|---|---|---|
| ID grouping | injections | injections | Frequency |
| A (n = 2) | 50 mL | 3 | 30 days/1 injection |
| B (n = 2) | 100 mL | 3 | 30 days/1 injection |
| C (n = 20 | 200 mL | 3 | 30 days/1 injection |

Six patients have been treated with intrathecal exosomes (UCF-E) (Table 16) and 3 out of 6 are the patients described in Table 10. These patients showed good improvement after exosome injections (UCF-E) and it is anticipated that a greater improvement will be achieved in higher dose patients.

TABLE 16
Patients treated with intrathecally administered
exosomes (UCF-E) every 14 days

| | Dose per | Total | |
|---|---|---|---|
| Grouping | injection | injections | Frequency |
| A (n = 3) | 400 μL | 3 | 14 days |
| C (n = 3) | 1600 μL | 3 | 14 days |

TABLE 17
Patients treated with intrathecally administered
exosomes (UCF-E) every 7 days

| | Dose per | Total | |
|---|---|---|---|
| Grouping | injection | injections | Frequency |
| A (n = 3) | 400 μL (low) | 3 | 7 days |
| B (n = 3) | 1600 μL (high) | 3 | 7 days |

After admission, hematoma was punctured and drained under robot, and rt-PA injected to dissolve the hematoma. After three days, most of the hematoma was drained without bleeding and other complications. Seven days after the operation, UCF-E was injected intrathecally. The injection volume was either 400 μL or 800 μl, and the injection was repeated on the 14^{th} and 21^{st} days after the operation.

TABLE 18
Patient information

| | Group A | | | Group B | | |
|---|---|---|---|---|---|---|
| | No. | No. | No. | No. | No. | No. |
| Groups | 1 | 2 | 3 | 4 | 5 | 6 |

Basic information

| | | | | | | |
|---|---|---|---|---|---|---|
| Age | 54 | 47 | 46 | 40 | 52 | 59 |
| Gender | M | F | M | M | F | F |
| Hypertension history | Y | Y | Y | Y | Y | Y |
| Diabetes history | N | Y | N | Y | N | Y |
| Hyperlipidemia history | N | Y | N | Y | N | N |
| Antiplatelet/ | Y | N | N | N | N | Y |
| anticoagulant drugs | | | | | | |
| Dose group | low | low | low | high | high | high |

Clinical information

| | | | | | | |
|---|---|---|---|---|---|---|
| Location of hemorrhage | left | left | right | left | right | right |
| (Basal Ganglia) | | | | | | |
| Volume of hemorrhage(mm³) | 48 | 64 | 67 | 62 | 70 | 75 |
| Time from onset to injection | 8 | 7 | 8 | 9 | 7 | 10 |
| (days) | | | | | | |

TABLE 19
Functional evaluation after injections of UCF-E

| | | | | | |
|---|---|---|---|---|---|
| No. 1 | No. 2 | No. 3 | No. 4 | No. 5 | No. 6 |

NIHSS

| | | | | | | |
|---|---|---|---|---|---|---|
| Day 1 (after | 19 | 25 | 22 | 26 | 18 | 19 |
| hemorrhage) | | | | | | |
| Day 7 | 21 | 25 | 21 | 26 | 19 | 18 |
| Day 21 | 20 | 23 | 22 | 23 | 18 | 16 |
| Day 45 | 18 | 21 | 20 | 18 | 11 | 9 |
| Day 60 | 17 | 20 | 21 | 15 | 11 | 9 |

Barthel score

| | | | | | | |
|---|---|---|---|---|---|---|
| Day 1 | 36 | 23 | 38 | 28 | 35 | 37 |
| Day 7 | 36 | 23 | 36 | 28 | 34 | 37 |
| Day 21 | 42 | 25 | 36 | 36 | 41 | 56 |
| Day 45 | 46 | 37 | 42 | 52 | 59 | 66 |
| Day 60 | 47 | 39 | 49 | 65 | 63 | 75 |

Language impairment

| | | | | | | |
|---|---|---|---|---|---|---|
| Day 1 | Aphasia | Aphasia | — | Aphasia | — | — |
| Day 7 | Aphasia | Aphasia | — | Aphasia | — | — |
| Day 21 | Aphasia | Aphasia | — | Could say a | — | — |
| | | | | few words | | |
| Day 45 | Aphasia | Pronounce | — | Can form a | — | — |
| | | | | sentence | | |
| | | | | but spoke | | |
| | | | | slowly | | |
| Day 60 | Aphasia | Pronounce | — | Spoke | — | — |
| | | | | faster and | | |
| | | | | could sing | | |
| Affected upper | | | | | | |
| limb muscle strength | | | | | | |
| Day 1 | 0 | 0 | 0 | 0 | 0 | I |
| Day 7 | 0 | 0 | 0 | 0 | 0 | I |
| Day 21 | 0 | 0 | 0 | I | I | I |
| Day 45 | I | I | 0 | II | I | II |
| Day 60 | I | II | I | III | II | II |
| Affected lower | | | | | | |
| limb muscle strength | | | | | | |
| Day 1 | I | 0 | 0 | 0 | 0 | II |
| Day 7 | I | 0 | 0 | 0 | 0 | II |
| Day 21 | II | II | II | III | II | III |
| Day 45 | III | III | II | III | IV | IV |
| Day 60 | IV | IV | III | V | IV | IV |

UCF-E were injected intrathecally without complications in all six patients. No significant hemodynamic or pulmonary complications were observed. At the end of the follow up, there were no signs of re-bleeding or worsening of hemorrhagic infarction or cerebral hemorrhage. No seizure was observed in any patient. Low grade fever (38.5° C.) was noted in 5 patients from the study group within the first 24 h of injection. The fever in all 5 patients subsided within 2 days without specific pharmacological intervention.

In addition, a study on the effect of exosome preparations (UCF-E) is being performed in a rat model of hemorrhagic stroke according to the table below.

TABLE 20
Outline of rat hemorrhagic stroke study

| | Dose per | Total | |
|---|---|---|---|
| Grouping | injection | injections | Frequency |
| Control group (n = 3) | 20 μL | 3 | 3 days |
| UCF-E group (n = 3) | 20 μL | 3 | 3 days |
| UCF-E group (n = 3) | 80 μL | 3 | 3 days |
| Sham (n = 3) | — | — | — |

The extent of brain atrophy was assessed 36 days post injury in an additional study. The degree of brain atrophy in the UCF-E group was significantly lower than that in the saline control group (p=0:0893; FIG. 12), indicating a neuroprotective effect in the UCF-E treated group.

### 8. A Human Case Study

TABLE 21
Patient information
Demographic information

| Age | 40 | |
|---|---|---|
| Gender | M | |
| Weight | 210 | kg |
| Height | 2.09 | meters |

Clinical information

| Consciousness on admission | Shallow coma | |
|---|---|---|
| Medical history | Hypertension Hyperlipidemia | |
| Location of hemorrhage | Left basal ganglia | |
| Broken into the ventricle | N | |
| Blood loss | 62 | mm³ |

| | |
|---|---|
| Symptoms and signs | Lost speech function. |
| | Muscle strength of right limb: Level 0. |
| | Glasgow Coma Scale score: 9. |

### Treatment Process

After admission, hematoma puncture and drainage under the guidance of a robot was performed, and rt-PA was injected to dissolve the hematoma. After re-examination three days later, the hematoma was essentially drained clean, and there were no complications such as re-bleeding. UCF-E was administered by intrathecal injection into the L3-4 intervertebral space seven days after surgery, and the injection dose of this patient was 1600 μL. After releasing the same amount of cerebrospinal fluid, exosomes were injected directly into the intrathecal area at a speed of 400 μL per minute for a total of 4 minutes to complete the injection. Regular rehabilitation exercises were performed after.

TABLE 22
Functional evaluation

| | Day 1 | Day 7 | Day 21 | Day 45 | Day 60 |
|---|---|---|---|---|---|
| NIHSS score | 19 | 18 | 16 | 11 | 11 |
| Barthel score | 28 | 31 | 39 | 45 | 57 |
| Consciousness | Light | Clear | Clear mind | Clear mind | Clear mind |
| | consciousness/ | mind | | | |
| | coma | | | | |
| Swallowing | Nasal | Nasal | Nasal feeding | Liquid feeding | Essentially |
| | feeding | feeding | | | normal diet |
| Speech | none | none | Can speak in | Can speak | Tone started |
| | | | sentences but | consecutively | to change and |
| | | | speed is slow | | could sing |
| | | | (inconsecutive) | | |
| Right upper | 0 | 0 | 1 | 2 | 3 |
| limb muscle | | | | | |
| strength | | | | | |
| Right lower | 0 | 0 | 1 | 3 | 4 |
| limb muscle | | | | | |
| strength | | | | | |
| Walking | In bed, | In bed, | Unable to walk | Can walk with | Speed, frequency |
| | unable to | unable to | by himself but | help of others | and leg lifting |
| | walk by | walk by | could stand | with difficulty | height when walking |
| | himself | himself | alone | and long breaks, | significantly |
| | and unable | and unable | | leg lifting range | increased, and |
| | to stand | to stand | | was small, and | hand grip strength |
| | | | | only had subtle | increased |
| | | | | hand movement | |

What is claimed is:

1-35. (canceled)

36. A method of treating or improving a symptom of a neuronal injury in a subject, the method comprising administering to the subject a therapeutically effective amount of an acellular allogenic preparation, selected from acellular human umbilical cord plasma with exosomes (UCF) or exosomes only (UCF-E) from human umbilical cord plasma, wherein the UCF or UCF-E is derived from a human umbilical cord blood, wherein the neuronal injury is ischemic brain injury, haemorrhagic brain injury, or traumatic brain injury, and wherein the UCF or UCF-E is first administered from 3 to 90 days after the neuronal injury.

37. The method of claim 36, wherein the UCF or UCF-E is first administered 7 to 10 days after the neuronal injury.

38. The method of claim 36, wherein the UCF or UCF-E is administered intrathecally.

39. The method of claim 36, wherein the UCF or UCF-E is administered periodically to the subject, wherein the period of administration is every week, every two weeks, every three weeks, every month, or a combination thereof or wherein the period of administration is daily, every 5 days, every 10 days, every 15 days, every 20 days, every 25 days, about every 30 days, or a combination thereof.

40. The method of claim 36, wherein the ischemic brain injury or haemorrhagic brain injury is a stroke.

41. The method of claim 36, wherein an immunosuppressive agent is not administered.

42. The method of claim 36, wherein the acellular human umbilical cord plasma with exosomes (UCF) is characterized by a level of one or more of GM-CSF, HGF, MIP-1b (CCL4) and PDGF-bb that is at least 20% higher than is found in normal adult serum.

43. The method of claim 36, wherein the acellular allogenic preparation with exosomes (UCF) and exosomes only (UCF-E) are characterized by a level of IP-10 (CxCL10) that is at least 60% of the level found in normal adult serum.

44. The method of claim 36, wherein exosomes in the allogenic preparation containing exosomes (UCF or UCF-E) comprise at least one of CD63 and TSG101 and lack lipoprotein markers APOB and APOE.

45. The method of claim 36, wherein the acellular allogenic preparation containing exosomes (UCF and UCF-E) comprises exosomes with a mean, median or mode particle size of about 30-150 nm.

46. The method of claim 36, wherein the method comprises administering the acellular allogenic preparation with exosomes (UCF and UCF-E) a total of two, three, four, five, six, seven, eight, nine or ten times.

47. The method of claim 36, wherein the symptom is selected from dystonia, aphasia, infarct size, inflammation, systemic inflammation, loss of white matter, loss of grey matter, apoptotic cells in the peri-infarct region, and CD16/CD32 positive cells in the peri-infarct region.

48. The method of claim 36, wherein the method further comprises administering the allogenic preparation with exosomes (UCF and UCF-E) with at least one additional therapeutic agent.

49. The method of claim 48, wherein the additional therapeutic agent is an anti-inflammatory agent, steroid, dipyridamole, astaxanthin, dabigatran, losartan, nimodipine, policosanol, rivaroxaban, ticlopidine, mannitol, intracarotid arterial hyperosmolar mannitol (ICAHM), RMP-7, or regadenoson.

50. A method of improving a mRS score or NIHSS score of a subject with an ischemic brain injury, the method comprising administering to the subject a therapeutically effective amount of an acellular allogenic preparation with exosomes (UCF or UCF-E), wherein the UCF or UCF-E is derived from pooled human umbilical cord blood, wherein the pooled blood is from umbilical cords with the same blood group or from umbilical cords with different blood groups, and wherein the UCF or UCF-E is first administered from 3 to 90 days after the ischemic brain injury.

51. The method of claim 50, wherein an immunosuppressive agent is not administered.

52. The method of claim 50, wherein the allogenic preparation with exosomes (UCF or UCF-E) is administered periodically to the subject, wherein the period of administration is every week, every two weeks, every three weeks, every month, or a combination thereof or wherein the period of administration is daily, every 5 days, every 10 days, every 15 days, every 20 days, every 25 days, about every 30 days, or a combination thereof.

53. The method of claim 50, wherein the method comprises administering the acellular allogenic preparation with exosomes (UCF or UCF-E) a total of two, three, four, five, six, seven, eight, nine or ten times.

54. The method of claim 36, wherein the UCF or UCF-E is derived from blood of a single umbilical cord, or pooled blood from multiple umbilical cords, wherein the pooled blood is from umbilical cords with the same blood group or from umbilical cords with different blood groups.
